(19) United States
(12) Patent Application Publication
(10) Pub. No.: US 2025/0152580 A1
(43) Pub. Date: May 15, 2025

(54) METHODS FOR TREATING IMMUNE DISEASES

(71) Applicants: Ascentage Pharma (Suzhou) co., Ltd., Suzhou, Jiangsu (CN); Ascentage Pharma Group Corp Limited, Hong Kong (CN)

(72) Inventors: Yifan ZHAI, Suzhou, Jiangsu (CN); Dajun YANG, Suzhou, Jiangsu (CN); Lei YANG, Suzhou, Jiangsu (CN); Douglas Dong FANG, Suzhou, Jiangsu (CN); Yanhui KONG, Suzhou, Jiangsu (CN)

(21) Appl. No.: 18/838,451

(22) PCT Filed: Mar. 6, 2023
(86) PCT No.: PCT/CN2023/079837

§ 371 Date: Aug. 14, 2024

(30) Foreign Application Priority Data
Mar. 7, 2022 (WO) PCT/CN2022/079595

(51) Int. Cl. A61K 31/496; A61K 31/444; A61K 31/675; A61P 1/00; A61P 11/06; A61P 17/00; A61P 17/06

(52) U.S. Cl. CPC A61K 31/496; A61K 31/444; A61K 31/675; A61P 1/00; A61P 11/06; A61P 17/00; A61P 17/06

(57) ABSTRACT

Methods for treating immune diseases, specifically methods for the prevention and/or inhibition of autoimmune response or overactive immune response, comprising administering to the subject an effective amount of a compound of formula I, X or XI, or a pharmaceutically acceptable salt or solvate thereof. Methods for the prevention and/or inhibition of autoimmune disease, allergic disease or immune-mediated inflammatory disease, comprising administering to the subject an effective amount of a compound of formula I, X or XI, or a pharmaceutically acceptable salt or solvate thereof.

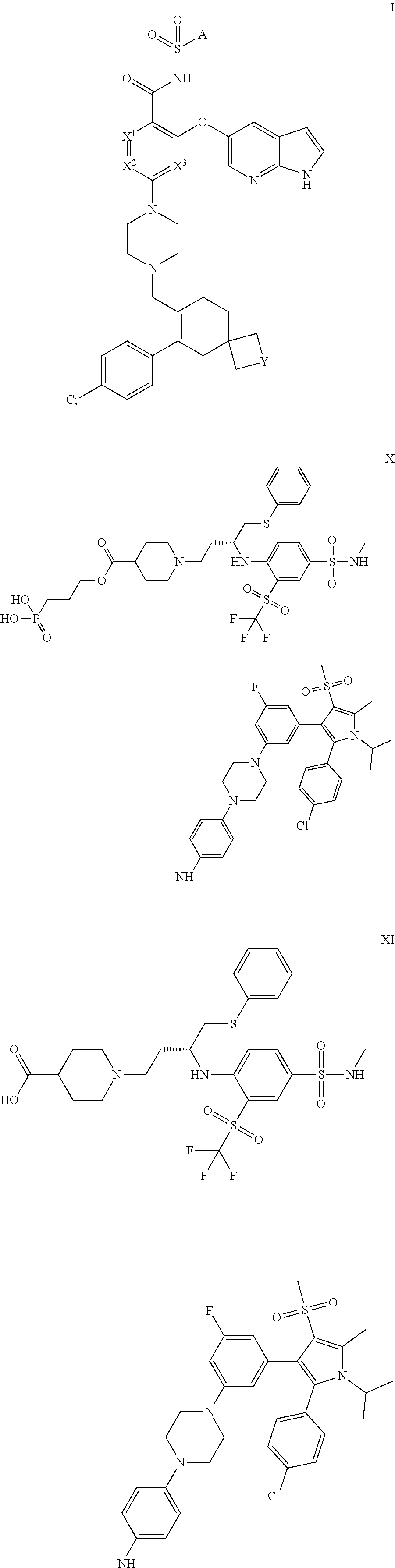

### RELATED APPLICATIONS

This application is a U.S. National Phase application, filed under U.S.C. § 371, of International Application No. PCT/CN2023/079837, filed Mar. 6, 2023, which claims priority to, and the benefit of, Chinese Application No. PCT/CN2022/079595, filed Mar. 7, 2022, the entire contents of each of which are incorporated herein by reference in their entireties.

### FIELD OF DISCLOSURE

The present disclosure relates to methods for treating immune diseases.

### BACKGROUND

The immune system is made up of two parts: the innate (general) immune system and the adaptive (specialized) immune system. The innate and adaptive immune systems converge into 3 major kinds of cell mediated effector immunity, which are categorized as type 1, type 2, and type 3. Type 1 immunity consists of T-bet⁺ IFN-g-producing group 1 ILCs (ILC1 and natural killer cells), CD8⁺ cytotoxic T cells (T_{C}1), and CD4⁺ Tₕ1 cells, which protects against intracellular microbes through activation of mononuclear phagocytes. Type 2 immunity consists of GATA-3⁺ ILC2s, T_{C}2 cells, and Tₕ2 cells producing IL-4, IL-5, IL-13, etc., which induces mast cell, basophil, and eosinophil activation, as well as IgE antibody production, thus protecting against helminthes, venoms and repairing tissue injury. Type 3 immunity is mediated by retinoic acid-related orphan receptor γδ⁺ ILC3s, T_{C}17 cells, and T_{H}17 cells producing IL-17, IL-22, etc., which recruits neutrophils and induce epithelial antimicrobial responses, thus protecting against extracellular bacteria and fungi (Annunziato F, Romagnani C, Romagnani S. The 3 major types of innate and adaptive cell-mediated effector immunity, Journal of Allergy & Clinical Immunology, 2015, 135(3): 626-635). Dysregulation of type 1 and type 3 immunity mediates autoimmune diseases. Dysregulation of type 2 response can cause allergic diseases.

IBD (inflammatory bowel disease), classically divided into Crohn's disease (CD) and ulcerative colitis (UC), is a chronic, debilitating condition characterized by relapsing and remitting episodes of gastrointestinal (GI) inflammation. CD affects the superficial mucosa, starting with the rectum, in a continuous pattern and is limited to the colon. UC is characterized by transmural inflammation that can affect any part of the GI tract from mouth to anus. Acute DSS colitis is caused primarily by disruption of the epithelium and activation of macrophages and neutrophils, which can be induced with the absence of adaptive immunity, so it is mainly recognized as an innate immune induced model. Intrarectal acute TNBS administration to mice induces a transmural colitis mainly driven by a type 1 immune response and characterized by infiltration of the lamina propria with CD4⁺ T cells, neutrophils, and macrophages. Adoptive transfer of naïve CD4⁺ T cells (CD4⁺CD45RB^{high} T cells) from donor mice into syngeneic immunodeficient (lymphopenic) SCID or Rag1^{-/-} recipient mice cause a wasting disease and a primarily colonic inflammation that develops 5 to 10 weeks after treatment. Type 3 (T_{H}17) immune responses appear to be dominant although both T_{H}1 and T_{H}17 responses result in cells producing IFN-γ in this adoptive T transfer IBD model. (Kiesler P, Fuss I J, Strober W. Experimental Models of Inflammatory Bowel Diseases. Cell Mol Gastroenterol Hepatol. 2015, 1(2): 154-170).

AD (atopic dermatitis) is a chronic and relapsing inflammatory skin disease of increasing prevalence, especially in industrialized countries and is a disease arising from immunological dysfunction. AD significantly impairs a patient's quality of life with severe pruritus as a major issue that impairs sleep and contributes to major psychological disturbances.

Asthma is a chronic inflammatory disease characterized by narrowing of the airways which affects millions of people worldwide, and the number of affected individuals continues to increase.

OVA induced asthma and OXA induced atopic dermatitis (AD) are typical models of type 2 immune responses. Elevated lung eosinophils and serum immunoglobulin E (IgE) are two key causes of these type 2 inflammation diseases (Akdis C A, Arkwright P D, Brüggen M C, Busse W, Gadina M, Guttman-Yassky E, Kabashima K, Mitamura Y, Vian L, Wu J, Palomares O. Type 2 immunity in the skin and lungs. Allergy. 2020, 75(7): 1582-1605).

Psoriasis is a chronic skin disease characterized by circumscribed red patches covered with white scales. There is no permanent cure for psoriasis and treatments aim at reducing symptoms such as pain, inflammation, and scaling. Participation of IL-23-Th17 axis in IMQ psoriasis model demonstrates this model driven by the type 3 immune response. IL-23 promotes the development of Th17 cells and the resulting production of cytokines such as IL-17A, IL-17F, and IL-22, all which are involved in mediating psoriasiform changes. (Li B, Huang L, Lv P, Li X, Liu G, Chen Y, Wang Z, Qian X, Shen Y, Li Y, Fang W. The role of Th17 cells in psoriasis. Immunol Res. 2020, 68(5): 296-309).

Inhalation of LPS in vivo can induce excessive infiltration of neutrophils in lung, and dysregulated infiltration of neutrophils into the lung is a key pathogenic factor for a series of lung inflammation related diseases, such as chronical obstructive pulmonary disease, asthma, etc. (Korsgren M, Linden M, Entwistle N, et al. Inhalation of LPS induces inflammatory airway responses mimicking characteristics of chronic obstructive pulmonary disease. Clin Physiol Funct Imaging. 2012; 32(1):71-79.)

Hypersensitivity reactions are exaggerated or inappropriate immunologic responses occurring in response to an antigen or allergen. Type IV hypersensitivity reaction, known as delayed type hypersensitivity (DTH) reaction, is mediated by T cells that provoke an inflammatory reaction against exogenous or endogenous antigens. Delayed-type hypersensitivity (DTH) is a useful approach for evaluating type 1 immune responses (Allen I C. Delayed-type hypersensitivity models in mice. Methods Mol Biol. 2013, 1031:101-7).

There is still a high unmet need to develop novel therapeutic options for inflammatory diseases.

### SUMMARY

In one aspect, the present disclosure provides a method for the prevention and/or inhibition of autoimmune response or overactive immune response in a subject, comprising administering to the subject an effective amount of a therapeutic agent, wherein the therapeutic agent is a compound of formula I, X or XI, or a pharmaceutically acceptable salt or solvate thereof:

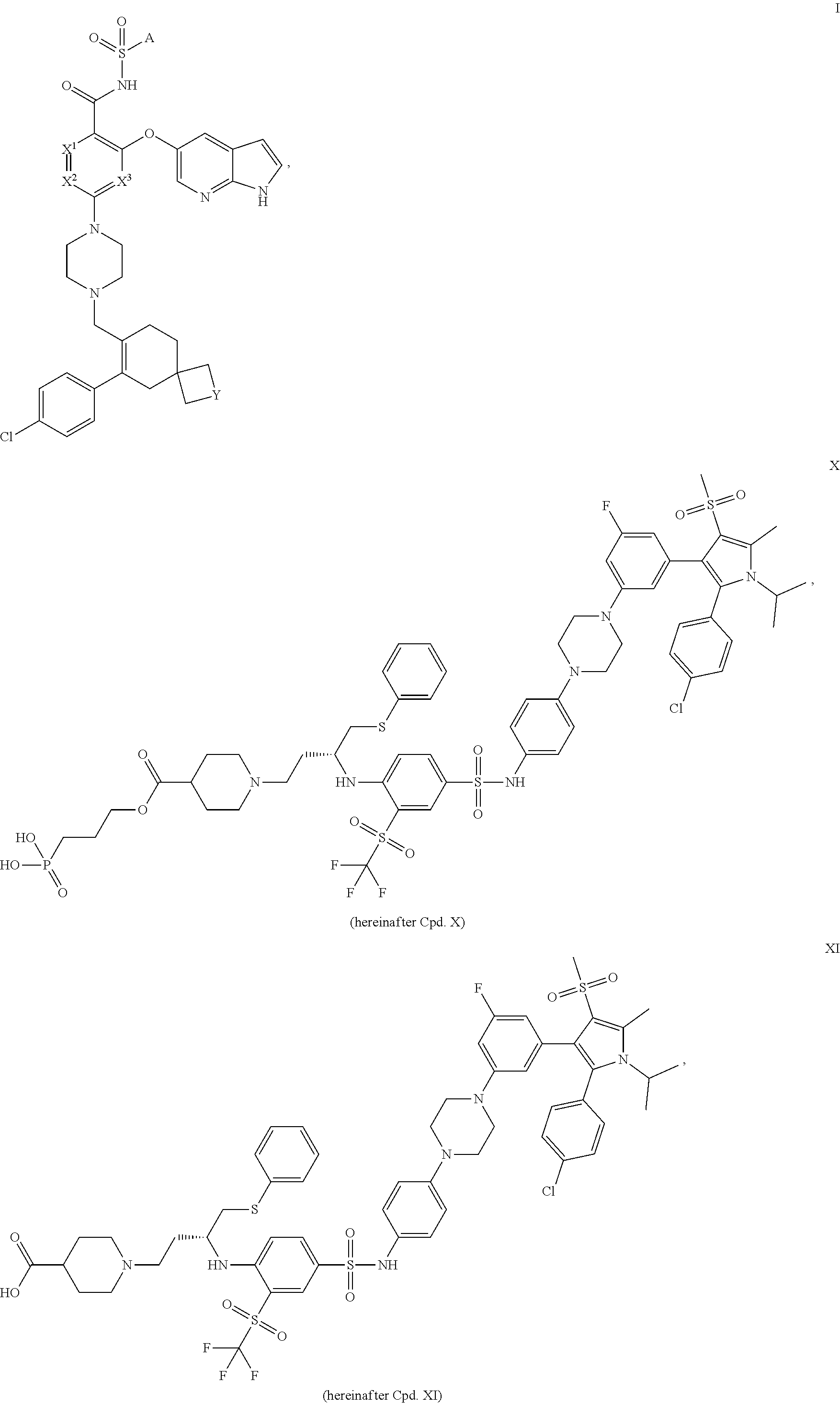

wherein, in the formula I,
A is

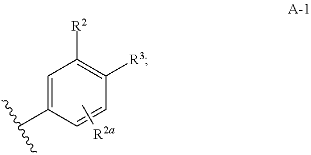

X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and
Y selected from the group consisting of —CH₂— and —O—.

In another aspect, the present disclosure provides a use of the aforementioned therapeutic agent in the manufacture of a medicament for the prevention and/or inhibition of autoimmune response or overactive immune response in a subject.

In another aspect, the present disclosure provides a pharmaceutical composition for the prevention and/or inhibition of autoimmune response or overactive immune response, comprising the aforementioned therapeutic agent and a pharmaceutical acceptable excipient.

In some embodiments, the autoimmune response or overactive immune response is characterized by below feature a), b), c), d) or a combination thereof:
a) elevated neutrophils, monocytes, macrophages and/or natural killer cells;
b) elevated activated Th1 cells, and/or effector cells (e.g., monocytes, macrophages and NK cells);
c) elevated eosinophils and/or IgE production;
d) elevated activated Th17 cells and/or neutrophils.

In some embodiments, the autoimmune response or overactive immune response is characterized by below feature a), b), c), d) or a combination thereof:
a) elevated neutrophils, macrophages and/or natural killer cells in lymph nodes, and/or elevated blood neutrophils and/or blood monocytes, and/or elevated neutrophils in lung;
b) elevated activated Th1 cells, and/or elevated effector cells (e.g., macrophages and NK cells) in lymph nodes;
c) elevated lung eosinophils and/or serum IgE production;
d) elevated activated Th17 cells and/or elevated neutrophils in lymph node and/or elevated neutrophils in blood.

In some embodiments, the subject with autoimmune response or overactive immune response has an immune disease, wherein the immune disease is autoimmune disease, allergic disease or immune-mediated inflammatory disease.

In another aspect, the present disclosure provides a method for the prevention and/or treatment of an immune disease in a subject, comprising administering to the subject an effective amount of the aforementioned therapeutic agent, wherein the immune disease is autoimmune disease, allergic disease or immune-mediated inflammatory disease.

In another aspect, the present disclosure provides a use of the aforementioned therapeutic agent in the manufacture of a medicament for the prevention and/or treatment of an immune disease, wherein the immune disease is autoimmune disease, allergic disease or immune-mediated inflammatory disease.

In another aspect, the present disclosure provides a pharmaceutical composition for the prevention and/or treatment of an immune disease, comprising the aforementioned therapeutic agent and a pharmaceutical acceptable excipient, wherein the immune disease is autoimmune disease, allergic disease or immune-mediated inflammatory disease.

In some embodiments, the immune disease is autoimmune disease.

In some embodiments, the immune disease is allergic disease.

In some embodiments, the immune disease is immune-mediated inflammatory disease.

In some embodiments, the immune disease excludes multiple sclerosis (MS) and systemic lupus erythematosus (SLE).

In some embodiments, the immune disease is selected from the group consisting of acute disseminated encephalomyelitis (ADEM), Addison disease, ankylosing spondylitis, antiphospholipid syndrome (APGS), aplastic anemia, autoimmune haemolytic anaemia (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenia purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, inflammatory bowel disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, Takayasu arthritis, Vogt-Kovangai-Harada disease, asthma, atopic dermatitis, allergic rhinitis, food allergy, acute urticaria, and Contact dermatitis.

In some embodiments, the immune disease is inflammatory bowel disease, atopic dermatitis, asthma, psoriasis, or Type IV hypersensitivity disease.

In some embodiments, the immune disease is inflammatory bowel disease. In some embodiments, the inflammatory bowel disease is Crohn's disease or ulcerative colitis.

In some embodiments, the immune disease is atopic dermatitis.

In some embodiments, the immune disease is asthma.

In some embodiments, the immune disease is psoriasis.

In some embodiments, the immune disease is related to excessive infiltration of neutrophils in lung, such as chronic obstructive pulmonary disease.

In some embodiments, the immune disease is Type IV hypersensitivity disease.

In some embodiments, the therapeutic agent can prevent and/or treat the immune disease by preventing and/or inhibiting feature a), b), c), d) or a combination thereof:
a) elevated neutrophils, monocytes, macrophages and/or natural killer cells;
b) elevated activated Th1 cells, and/or effector cells (e.g., monocytes, macrophages and NK cells);
c) elevated eosinophils and/or IgE production;
d) elevated activated Th17 cells and/or neutrophils.

In some embodiments, the therapeutic agent can prevent and/or treat the immune disease by preventing and/or inhibiting below feature a), b), c), d) or a combination thereof:
a) elevated neutrophils, macrophages and/or natural killer cells in lymph nodes, and/or elevated blood neutrophils and/or blood monocytes, and/or elevated neutrophils in lung;
b) elevated activated Th1 cells, and/or elevated effector cells (e.g., macrophages and NK cells) in lymph nodes;
c) elevated lung eosinophils and/or serum IgE production;
d) elevated activated Th17 cells and/or elevated neutrophils in lymph node and/or elevated neutrophils in blood.

### BRIEF DESCRIPTION OF THE DRAWING

FIG. 1 shows Cpd. 4 (preventive and therapeutic) significantly reduced DAI and improved body weight loss in TNBS induced mice IBD model. Two-way ANOVA, *** p<0.001, compared with Vehicle.

FIG. 2 shows Cpd. 4 (preventive and therapeutic) significantly decreased colon weight and increased colon length in TNBS induced mice IBD model. One-way ANOVA, **, p<0.01; *** p<0.001, compared with Vehicle.

FIG. 3 shows Cpd. 4 (preventive and therapeutic) significantly decreased neutrophils and monocytes and improved anemia in peripheral blood in TNBS induced mice IBD model. One-way ANOVA, **, p<0.01; *** p<0.001, compared with Vehicle.

FIG. 4 shows that Cpd. 4 significantly reduced neutrophils, NK cells, activated NK cells, Th1 (INF-γ⁺CD4⁺) and Th17 (IL-17A⁺CD4⁺) cells and Macrophage cells in TNBS induced mice IBD model. One-way ANOVA, *, p<0.05; **, p<0.01; *** p<0.001, compared with Vehicle.

FIG. 5 shows that Cpd. 4 (preventive and therapeutic) significantly decreased pathological score in TNBS induced mice IBD model. One-way ANOVA, *** p<0.001, compared with Vehicle.

FIG. 6 shows that Cpd. 4 therapeutic treatment significantly reduced the fibrosis score in TNBS induced IBD mice model. *** p<0.001, compared with Vehicle.

FIG. 7 shows that Cpd. 4 significantly reduced DAI score in CD4⁺CD45RB^{high} T transfer mice IBD model. Two-way ANOVA, *** p<0.001, compared with Vehicle.

FIG. 8 shows that Cpd. 4 significantly decreased colon weight and increased colon length in CD4⁺CD45RB^{high} T transfer mice IBD model. One-way ANOVA, * p<0.05; **, p<0.01; *** p<0.001, compared with Vehicle.

FIG. 9 shows that Cpd. 4 significantly decreased pathological score in CD4⁺CD45RB^{high} T transfer mice IBD model. One-way ANOVA, **, p<0.01; *** p<0.001, compare with Vehicle.

FIG. 10 shows that Cpd. 4 significantly reduced DAI score in DSS induced mice IBD model. Two-way ANOVA, *** p<0.001, compared with Vehicle.

FIG. 11 shows that Cpd. 4 significantly decreased colon weight and increased colon length in DSS induced mice IBD model. One-way ANOVA, * p<0.05; **, p<0.01; *** p<0.001, compared with Vehicle.

FIG. 12 shows that Cpd. 4 significantly decreased pathological score in DSS induced mice IBD model. One-way ANOVA, *** p<0.005, compared with Vehicle.

FIG. 13 shows that Cpd. 4 and Cpd. X significantly reduced airway stenosis index in OVA-induced mouse asthma model. One-way ANOVA, *** p<0.001, compared with Vehicle.

FIG. 14 shows that Cpd. 4 and Cpd. X treatment significantly downregulated percentage of eosinophils in BALF in OVA-induced asthma model. One-way ANOVA, **, p<0.01; *** p<0.001, compared with Vehicle.

FIG. 15 shows that Cpd. 4 significantly decreased IgE production in OVA-induced asthma model. One-way ANOVA, * p<0.05; *** p<0.001, compared with Vehicle.

FIG. 16 shows that Cpd. 4 and Cpd. X significantly ameliorated pathological score in lung tissues in OVA-induced asthma model. One-way ANOVA, *** p<0.001, compared with Vehicle.

FIG. 17 shows that Cpd. 4 significantly reduced ear thickness in OXA induced atopic dermatitis model mice. One-way ANOVA, *** p<0.001, compared with Vehicle.

FIG. 18 shows that Cpd. 4 significantly reduced PASI score in IMQ-induced psoriasis model mice. One-way ANOVA, *** p<0.001, compared with Vehicle.

FIG. 19 shows that Cpd. 4 significantly reduced ear thickness in IMQ-induced psoriasis model mice. One-way ANOVA, *** p<0.001, compared with Vehicle.

FIG. 20 shows that Cpd. 4 significantly reduced ear thickness in IL-23-induced mice psoriasis model. One-way ANOVA, *** p<0.001, compared with Vehicle.

FIG. 21 shows that Cpd. 4, Cpd. X and Cpd. XI significantly reduced infiltration of BALF neutrophils in lung in a LPS inhalation model. One-way ANOVA, *** p<0.001, compared with Vehicle.

FIG. 22 shows that Cpd. 4 moderately reduced the increase of ear thickness in OXA-induced DTH model mice.

### DETAILED DESCRIPTION

Unless defined otherwise, all technical and scientific terms used herein have the same meaning as commonly understood by one of ordinary skill in the art to which this disclosure belongs.

The term “comprises” refers to “includes, but is not limited to”.

As used herein, the terms “preventing”, “prevention” refer to prophylactic administration to healthy patients to prevent the development of the diseases mentioned herein. Moreover, the term “preventing” means prophylactic administration to patients being in a pre-stage of the diseases to be treated.

As used herein, the terms “treating”, “treatment” refer to therapeutic therapy. When referring to a particular condition, the treatment refers to: (1) alleviating one or more of the biological manifestations of a disease or a condition, (2) interfering with (a) one or more points in the biological cascade that leads to a condition or (b) one or more of the biological manifestations of a condition, (3) improving one or more of symptoms, effects or side effects associated with a condition or one or more of the symptoms, effects or side effects associated with a condition or treatment thereof, or (4) slowing the progression of one or more of the biological manifestations of a disorder or a condition.

As used herein, the term “effective amount” refers to an amount that is effective to elicit the desired biological or medical response, including the amount of a therapeutic agent that, when administered to a subject for treating a disorder, is sufficient to effect such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

As used herein, the term “Type IV hypersensitivity disease” refers to any disease characterized by hypersensitivity reactions.

As used herein, the term “subject” to which administration is contemplated includes any animal (e.g., humans).

As used herein, the term “pharmaceutically acceptable” as used herein refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of compound 1 include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

As used herein, the use of the terms “a”, “an”, “the”, and similar referents in the context of describing the present disclosure (especially in the context of the claims) are to be construed to cover both the singular and the plural, unless otherwise indicated. The use of any and all examples, or exemplary language (including “e.g.”, “such as” and “for example”) provided herein, is intended to better illustrate the present disclosure and is not a limitation on the scope of the present disclosure unless otherwise claimed. No language in the specification should be construed as indicating any non-claimed element as essential to the practice of the present disclosure.

The term “halogen” refers to —Cl, —F, —Br, or —I.

The term “alkyl” as used by itself or as part of another group refers to the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl group is a straight chain C₁₋₆ alkyl group. In some embodiments, the alkyl group is a branched chain C₃₋₆ alkyl group. In some embodiments, the alkyl group is a straight chain C₁₋₄ alkyl group. In some embodiments, the alkyl group is a branched chain C₃₋₄ alkyl group. In some embodiments, the alkyl group is a straight or branched chain C₃₋₄ alkyl group. In some embodiments, the alkyl group is partially or completely deuterated, i.e., one or more hydrogen atoms of the alkyl group are replaced with deuterium atoms. Non-limiting exemplary C₁₋₄ alkyl groups include methyl, ethyl, propyl, isopropyl, butyl, sec-butyl, tert-butyl, and iso-butyl. Non-limiting exemplary C₁₋₄ groups include methyl, ethyl, propyl, isopropyl, and tert-butyl.

The term “optionally substituted alkyl” as used by itself or as part of another group refers to an alkyl that is unsubstituted or substituted with one, two, or three substituents independently selected from the group consisting of halo, nitro, cyano, hydroxy, alkoxy, amino, alkylamino, dialkylamino, and optionally substituted aryl. In one embodiment, the optionally substituted alkyl is substituted with two substituents. In some embodiments, the optionally substituted alkyl is substituted with one substituent. In some embodiments, the optionally substituted alkyl is unsubstituted. Non-limiting exemplary optionally substituted alkyl groups include —CH₂Ph, —CH₂CH₂NO₂, —CH₂CH₂OH, —CH₂CH₂OCH₃, and —CH₂CH₂F.

The term “halo” as used by itself or as part of another group refers to —Cl, —F, —Br, or —I.

The term “nitro” as used by itself or as part of another group refers to —NO₂.

The term “cyano” as used by itself or as part of another group refers to —CN.

The term “hydroxy” as used by itself or as part of another group refers to —OH.

The term “amino” as used by itself or as part of another group refers to —NH₂.

The term “haloalkyl” as used by itself or as part of another group refers to an alkyl substituted by one or more fluorine, chlorine, bromine and/or iodine atoms. In one embodiment, the alkyl group is substituted by one, two, or three fluorine and/or chlorine atoms. In some embodiments, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used by itself or as part of another group refers to an optionally substituted alkyl attached to a terminal oxygen atom. In one embodiment, the alkoxy group is a C₁₋₆ alkyl attached to a terminal oxygen atom. In some embodiments, the alkoxy group is a C₁₋₄ alkyl attached to a terminal oxygen atom. Non-limiting exemplary alkoxy groups include methoxy, ethoxy, and tert-butoxy.

The term “aryl” as used by itself or as part of another group refers to unsubstituted monocyclic or bicyclic aromatic ring systems having from six to fourteen carbon atoms, i.e., a C₆₋₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl

The term “optionally substituted aryl” as used herein by itself or as part of another group refers to an aryl that is either unsubstituted or substituted with one to five substituents independently selected from the group consisting of halo, nitro, cyano, hydroxy, alkyl, alkoxy, amino, alkylamino, dialkylamino, haloalkyl, and heterocyclo. In one embodiment, the optionally substituted aryl is an optionally substituted phenyl. In some embodiments, the optionally substituted phenyl has one substituent. In some embodiments, the optionally substituted phenyl is unsubstituted. Non-limiting exemplary substituted aryl groups include 2-methylphenyl, 2-methoxyphenyl, 2-fluorophenyl, and 4-chlorophenyl

The term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one embodiment, the cycloalkyl group has two rings. In one embodiment, the cycloalkyl group has one ring. In some embodiments, the cycloalkyl group is a C₃₋₈ cycloalkyl. In some embodiments, the cycloalkyl group is a C₃₋₆ cycloalkyl. In some embodiments, the cycloalkyl group is a C₃₋₅ cycloalkyl. The term “cycloalkyl” is meant to include groups wherein a ring —CH₂— is replaced with a —C(═O)—. Non-limiting exemplary cycloalkyl groups include cyclopropyl, cyclobutyl, cyclopentyl, cyclohexyl, cycloheptyl, cyclooctyl, norbornyl, decalin, imidazole, cyclohexenyl, cyclopentenyl, cyclopentanone, spiro[3.3]heptane, and bicyclo[3.3.1]nonane.

The term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. In one embodiment, the optionally substituted cycloalkyl is substituted with two substituents. In some embodiments, the optionally substituted cycloalkyl is substituted with one substituent. In some embodiments, the optionally substituted cycloalkyl is unsubstituted.

The term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. The term “heterocyclo” is meant to include cyclic ureido groups such as imidazolidinyl-2-one, cyclic amide groups such as β-lactam, γ-lactam, δ-lactam and ε-lactam, and cyclic carbamate groups such as oxazolidinyl-2-one. In one embodiment, the heterocyclo group is a 4-, 5-, 6-, 7- or 8-membered cyclic group containing one ring and one or two oxygen and/or nitrogen atoms. In one embodiment, the heterocyclo group is a 5- or 6-membered cyclic group containing one ring and one or two nitrogen atoms. In one embodiment, the heterocyclo group is an 8-, 9-, 10-, 11-, or 12-membered cyclic group containing two rings and one or two nitrogen atoms. In one embodiment, the heterocyclo group is a 4- or 5-membered cyclic group containing one ring and one oxygen atom. The heterocyclo can be optionally linked to the rest of the molecule through a carbon or nitrogen atom. Non-limiting exemplary heterocyclo groups include 1,4-dioxane, 2-oxopyrrolidin-3-yl, 2-imidazolidinone, piperidinyl, morpholinyl, piperazinyl, pyrrolidinyl, 8-azabicyclo[3.2.1]octane (nortropane), 6-azaspiro[2.5]octane, 6-azaspiro[3.4]octane, indolinyl, indolinyl-2-one, and 1,3-dihydro-2H-benzo[d]imidazole-2-one.

The term “optionally substituted heterocyclo” as used herein by itself or part of another group refers to a heterocyclo that is either unsubstituted or substituted with one, two, or three substituents independently selected from the group consisting of halo, nitro, cyano, hydroxy, alkyl, alkoxy, amino, alkylamino, dialkylamino, haloalkyl, and heterocyclo. Non-limiting exemplary optionally substituted heterocyclo groups include:

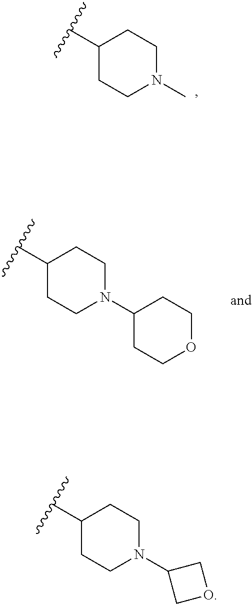

The term “(cycloalkyl)alkyl” as used by itself or as part of another group refers to an alkyl substituted with one optionally substituted cycloalkyl group. In one embodiment, the (cycloalkyl)alkyl is a C₁₋₄ alkyl substituted with one optionally substituted C₃₋₆ cycloalkyl. In one embodiment, the optionally substituted cycloalkyl group is substituted with a heterocyclo group. Non-limiting exemplary (cycloalkyl)alkyl groups include:

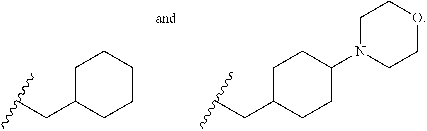 [SMILES: *CC1CCCCC1 ; "and" ; *CC1CCC(N2CCOCC2)CC1]

The term “alkylamino” as used by itself or as part of another group refers to —NHR¹⁰, wherein R¹⁰ is C₁₋₆ alkyl. In one embodiment, R¹⁰ is C₁₋₄ alkyl. Non-limiting exemplary alkylamino groups include —N(H)CH₃ and —N(H)CH₂CH₃.

The term “dialkylamino” as used by itself or as part of another group refers to —NR^{11a}R^{11b}, wherein R^{11a} and R^{11b} are each independently C₁₋₆ alkyl. In one embodiment, R^{11a} and R^{11b} are each independently C₁₋₄ alkyl. Non-limiting exemplary dialkylamino groups include —N(CH₃)₂ and —N(CH₃)CH₂CH(CH₃)₂.

The term “(heterocyclo)alkyl” as used by itself or as part of another group refers to an alkyl substituted with one optionally substituted heterocyclo group. In one embodiment, the (heterocyclo)alkyl is a C₁₋₄ alkyl substituted with one optionally substituted 4- to 6-membered heterocyclo group. The heterocyclo can be linked to the alkyl group through a carbon or nitrogen atom. Non-limiting exemplary (heterocyclo)alkyl groups include:

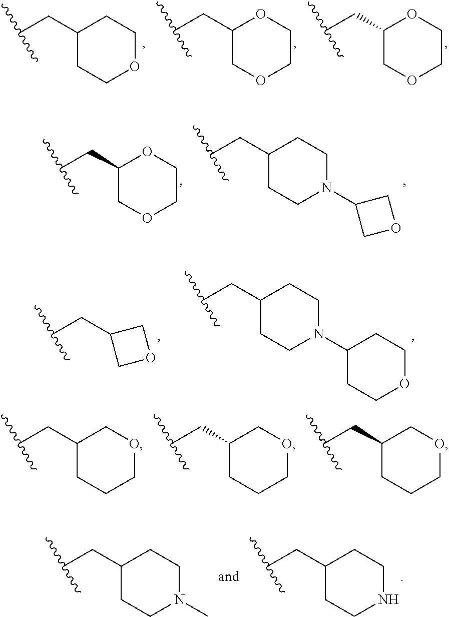

The term “heteroalkyl” as used by itself or part of another group refers to unsubstituted straight- or branched-chain aliphatic hydrocarbons containing from six to twelve chain atoms, i.e., 6- to 12-membered heteroalkyl, or the number of chain atoms designated, wherein at least two —CH₂— groups are independently replaced with —O—, —N(H)—, or —S—. The —O—, —N(H)—, or —S— can independently be placed at any interior position of the aliphatic hydrocarbon chain so long as each —O—, N(H)—, or —S— group is separated by at least two —CH₂— groups. In one embodiment, two —CH₂— groups are replaced with two —O— groups. In some embodiments, three —CH₂— groups are replaced with three —O— groups. Non-limiting exemplary heteroalkyl groups include —CH₂CH₂OCH₂CH₂OCH₃, —CH₂CH₂OCH₂CH₂N(H)CH₃, and —CH₂CH₂OCH₂CH₂OCH₂CH₂OCH₃.

In one aspect, the present disclosure provides a method for the prevention and/or inhibition of autoimmune response or overactive immune response in a subject, comprising administering to the subject an effective amount of a therapeutic agent, wherein the therapeutic agent is a compound of formula I, X or XI, or a pharmaceutically acceptable salt or solvate thereof:

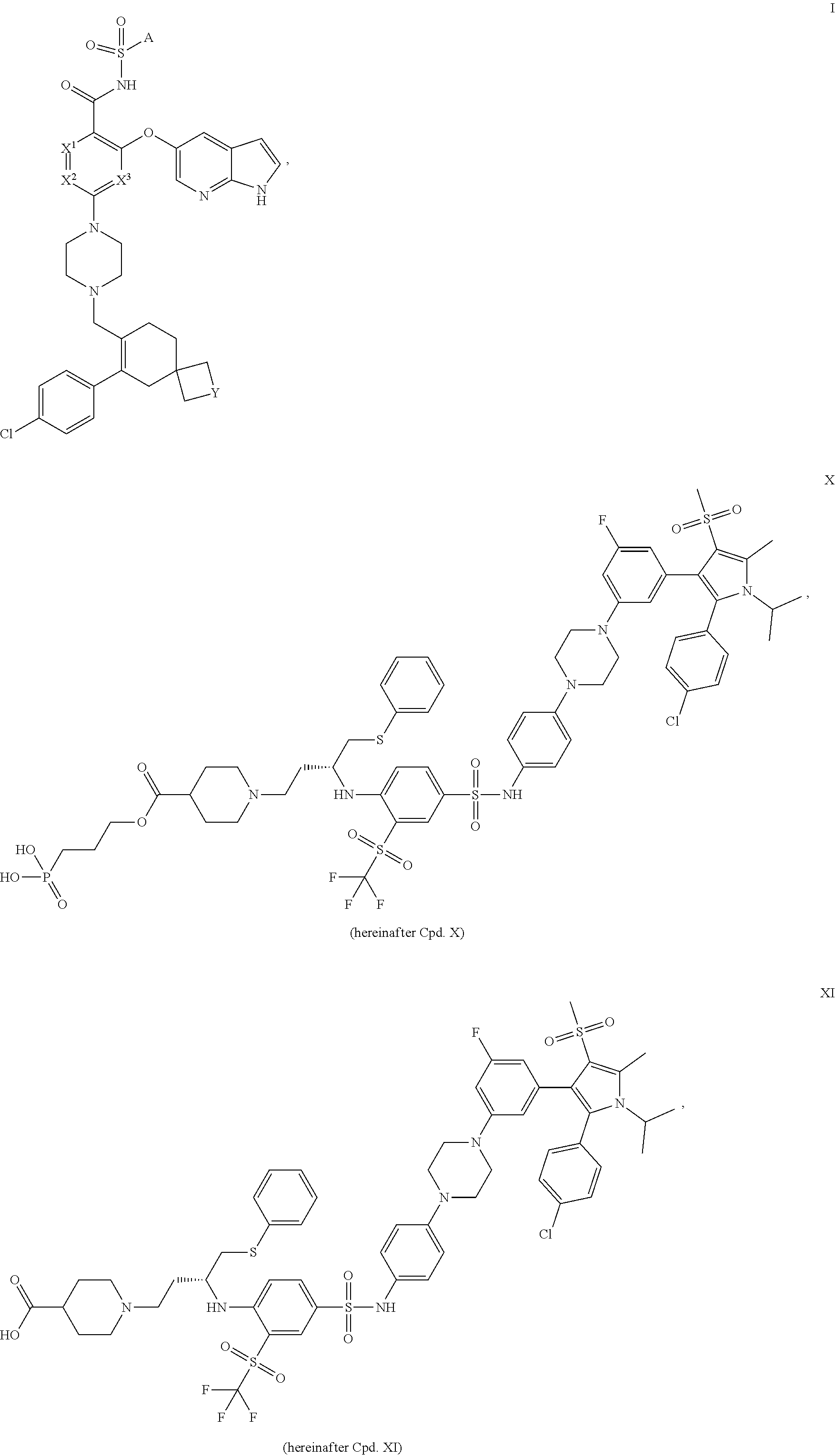

wherein, in the formula I,
A is

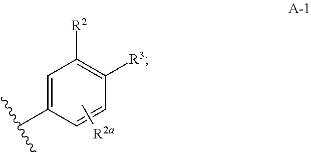

X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and

Y selected from the group consisting of —CH₂— and —O—.

In another aspect, the present disclosure provides a use of the aforementioned therapeutic agent in the manufacture of a medicament for the prevention and/or inhibition of autoimmune response or overactive immune response in a subject.

In another aspect, the present disclosure provides a pharmaceutical composition for the prevention and/or inhibition of autoimmune response or overactive immune response, comprising the aforementioned therapeutic agent and a pharmaceutical acceptable excipient.

In some embodiments, the autoimmune response or overactive immune response is characterized by below feature a), b), c), d) or a combination thereof:
a) elevated neutrophils, monocytes, macrophages and/or natural killer cells;
b) elevated activated Th1 cells, and/or effector cells (e.g., monocytes, macrophages and NK cells);
c) elevated eosinophils and/or IgE production;
d) elevated activated Th17 cells and/or neutrophils.

In some embodiments, the autoimmune response or overactive immune response is characterized by below feature a), b), c), d) or a combination thereof:
a) elevated neutrophils, macrophages and/or natural killer cells in lymph nodes, and/or elevated blood neutrophils and/or blood monocytes, and/or elevated neutrophils in lung;
b) elevated activated Th1 cells, and/or elevated effector cells (e.g., macrophages and NK cells) in lymph nodes;
c) elevated lung eosinophils and/or serum IgE production;
d) elevated activated Th17 cells and/or elevated neutrophils in lymph node and/or elevated neutrophils in blood.

In some embodiments, the subject with autoimmune response or overactive immune response has an immune disease, wherein the immune disease is autoimmune disease, allergic disease or immune-mediated inflammatory disease.

In another aspect, the present disclosure provides a method for the prevention and/or treatment of an immune disease in a subject, comprising administering to the subject an effective amount of the aforementioned therapeutic agent, wherein the immune disease is autoimmune disease, allergic disease or immune-mediated inflammatory disease.

In another aspect, the present disclosure provides a use of the aforementioned therapeutic agent in the manufacture of a medicament for the prevention and/or treatment of an immune disease, wherein the immune disease is autoimmune disease, allergic disease or immune-mediated inflammatory disease.

In another aspect, the present disclosure provides a pharmaceutical composition for the prevention and/or treatment of an immune disease, comprising the aforementioned therapeutic agent and a pharmaceutical acceptable excipient, wherein the immune disease is autoimmune disease, allergic disease or immune-mediated inflammatory disease.

In some embodiments, the immune disease is autoimmune disease.

In some embodiments, the immune disease is allergic disease.

In some embodiments, the immune disease is immune-mediated inflammatory disease.

In some embodiments, the immune disease excludes multiple sclerosis (MS) and systemic lupus erythematosus (SLE).

In some embodiments, the immune disease is selected from the group consisting of acute disseminated encephalomyelitis (ADEM), Addison disease, ankylosing spondylitis, antiphospholipid syndrome (APGS), aplastic anemia, autoimmune haemolytic anaemia (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenia purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, inflammatory bowel disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, Takayasu arthritis, Vogt-Kovangai-Harada disease, asthma, atopic dermatitis, allergic rhinitis, food allergy, acute urticaria, and Contact dermatitis.

In some embodiments, the immune disease is inflammatory bowel disease, atopic dermatitis, asthma, psoriasis, or Type IV hypersensitivity disease.

In some embodiments, the immune disease is inflammatory bowel disease. In some embodiments, the inflammatory bowel disease is Crohn's disease or ulcerative colitis.

In some embodiments, the immune disease is atopic dermatitis.

In some embodiments, the immune disease is asthma.

In some embodiments, the immune disease is psoriasis.

In some embodiments, the immune disease is related to excessive infiltration of neutrophils in lung, such as chronic obstructive pulmonary disease.

In some embodiments, the immune disease is Type IV hypersensitivity disease.

In some embodiments, the therapeutic agent can prevent and/or treat the immune disease by preventing and/or inhibiting feature a), b), c), d) or a combination thereof:
a) elevated neutrophils, monocytes, macrophages and/or natural killer cells;
b) elevated activated Th1 cells, and/or effector cells (e.g., monocytes, macrophages and NK cells);
c) elevated eosinophils and/or IgE production;
d) elevated activated Th17 cells and/or neutrophils.

In some embodiments, the therapeutic agent can prevent and/or treat the immune disease by preventing and/or inhibiting below feature a), b), c), d) or a combination thereof:
a) elevated neutrophils, macrophages and/or natural killer cells in lymph nodes and/or elevated blood neutrophils and/or blood monocytes, and/or elevated neutrophils in lung;
b) elevated activated Th1 cells, and/or elevated effector cells (e.g., monocytes, macrophages and NK cells) in lymph nodes;
c) elevated lung eosinophils and/or serum IgE production;
d) elevated activated Th17 cells and/or elevated neutrophils in lymph node and/or elevated neutrophils in blood.

In some embodiments, the aforementioned therapeutic agent is the compound of formula I, or a pharmaceutically acceptable salt or solvate thereof.

In some embodiments, the compound of formula I is a compound of formula II:

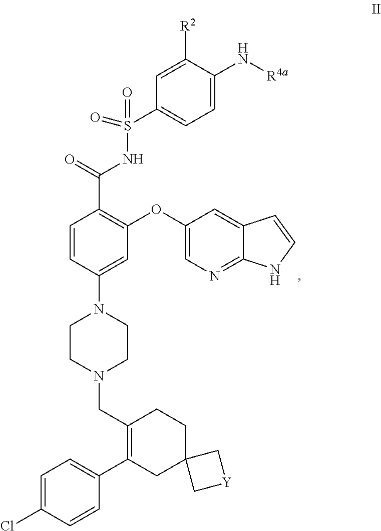

wherein Y is selected from the group consisting of —CH₂— and —O—; and
R² and R^{4a} are as defined above.

In some embodiments, the compound of formula I is a compound of formula III:

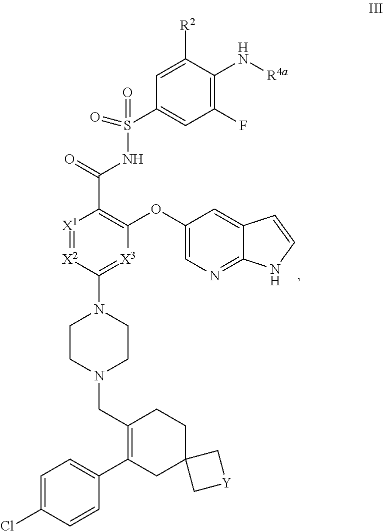

wherein Y is selected from the group consisting of —CH₂— and —O—; and X¹, X², X³, R², and R^{4a} are as defined above.

In some embodiments, in the formula of I or III, X¹, X², and X³ are each —CH═.

In some embodiments, in the formula of I or III, X¹ is —CF═, and X² and X³ are each —CH═.

In some embodiments, in the formula of I or III, X¹ and X³ are each —CH═, and X² is —CF═.

In some embodiments, in the formula of I or III, X¹ and X² are each —CH═, and X³ is —CF═.

In some embodiments, in the formula of I or III, X¹ is —N═, and X² and X³ are each —CH═.

In some embodiments, in the formula of I or III, X¹ and X³ are each —CH═, and X² is —N═.

In some embodiments, in the formula of I or III, X¹ and X² are each —CH═, and X³ is —N═.

In some embodiments, in the formula of I, II or III, Y is —O—.

In some embodiments, in the formula of I, II or III, Y is —CH₂—.

In some embodiments, in the formula of I, II or III, R² is —NO₂.

In some embodiments, in the formula of I, II or III, R^{4a} is selected from the group consisting of:

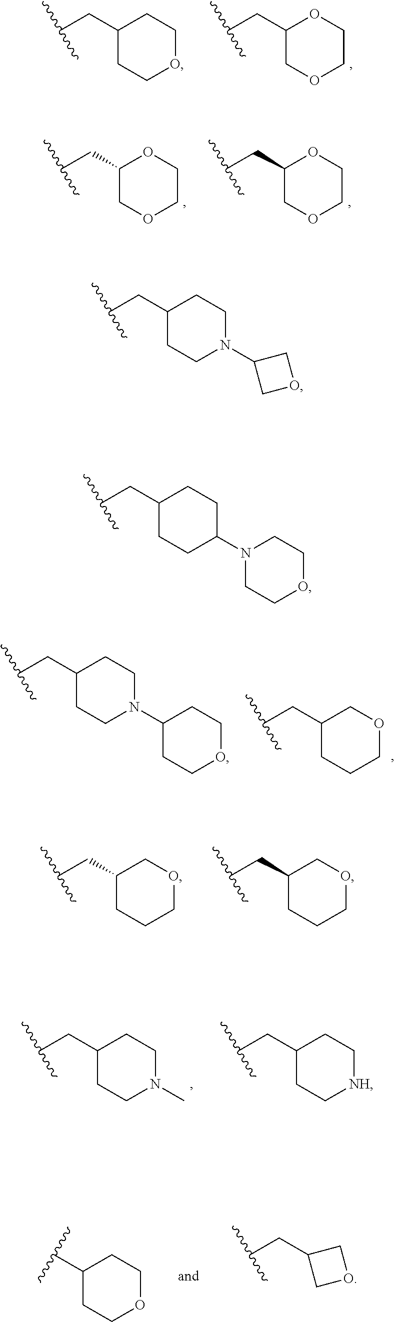

In some embodiments, in the formula of I, II or III, R^{4a} is selected from the group consisting of:

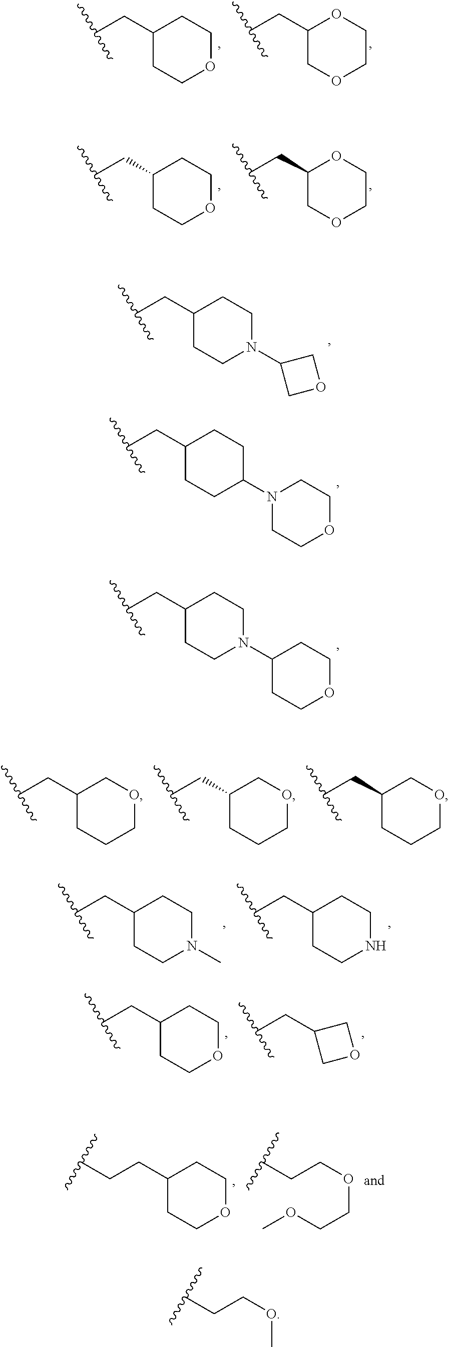

In some embodiments, the compound of formula I is a compound of formula IV;

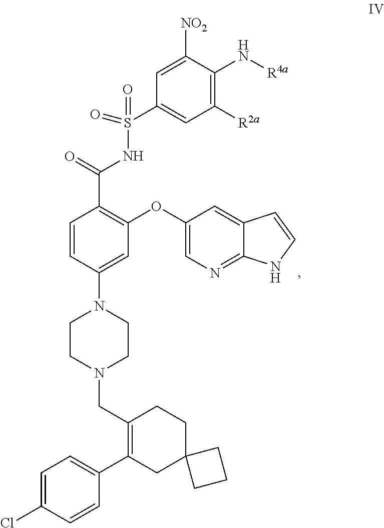

wherein R^{2a} is hydrogen or fluoro; and R^{4a} is as defined above.

In some embodiments, in the compound of formula IV, R^{4a} is selected from the group consisting of:

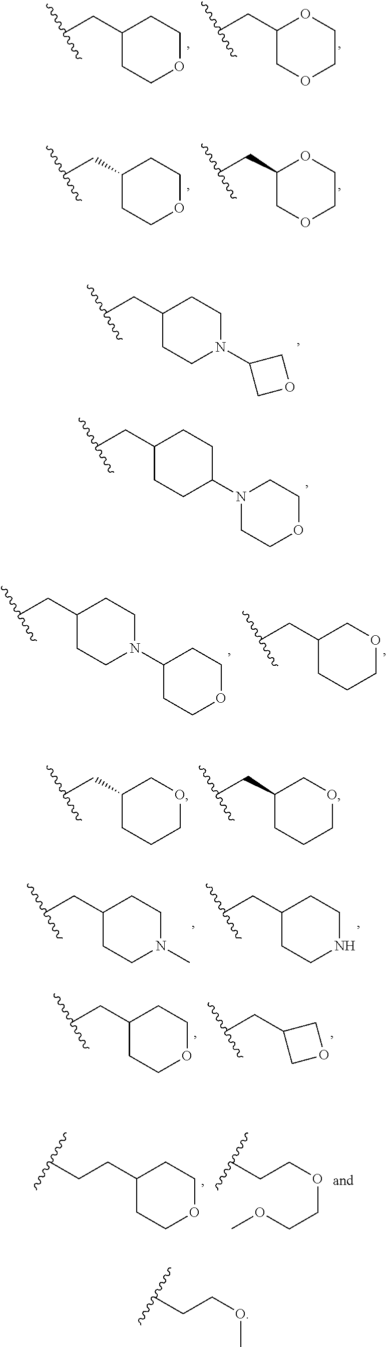

In some embodiments, the compound of formula I is a compound in the following Table 1.

TABLE 1

| Cpd. | | |
|---|---|---|
| No. | Structure | Name |
| 1 | 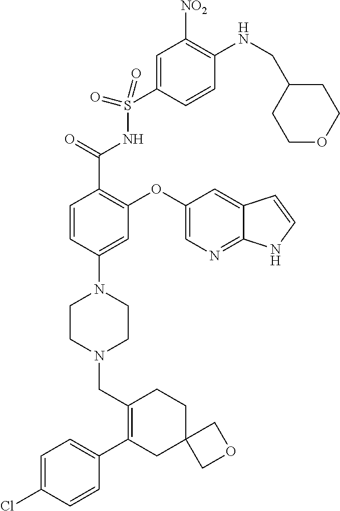 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)- 4-(4-((6-(4-chlorophenyl)-2- oxaspiro[3.5]non-6-en-7- yl)methyl)piperazin-1-yl)-N-((3-nitro-4- (((tetrahydro-2H-pyran-4- yl)methyl)amino)phenyl)sulfonyl)benzamide |
| 2 | 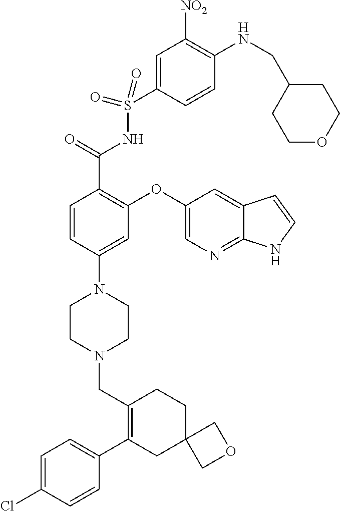 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-(((tetrahydro-2H-pyran-4- yl)methyl)amino)phenyl)sulfonyl)benzamide |
| 3 | 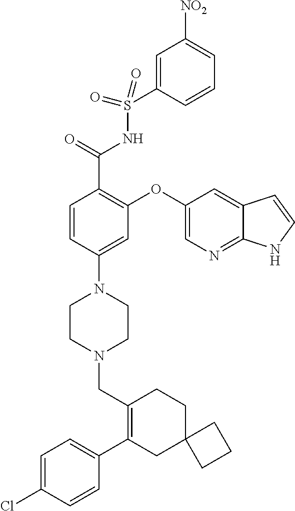 [SMILES: O=C(NS(=O)(=O)c1cccc([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitrophenyl)sulfonyl)benzamide |
| 4 | 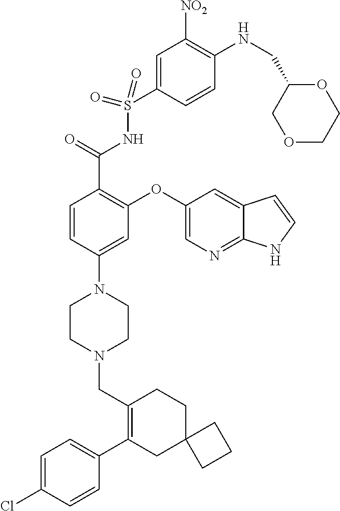 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)benzamide |
| 5 | 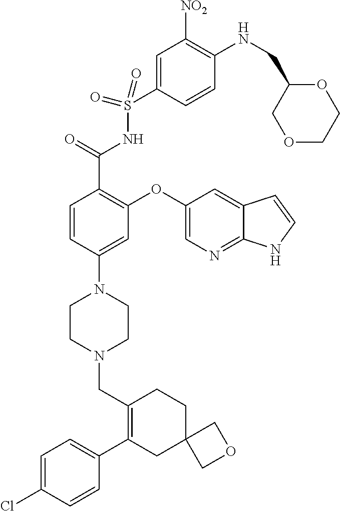 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CC3)COC4)CC2)cc1Oc1cnc2[nH]ccc2c1] | (R)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)-2-oxaspiro[3.5]non-6- en-7-yl)methyl)piperazin-1- yl)benzamide |
| 6 | 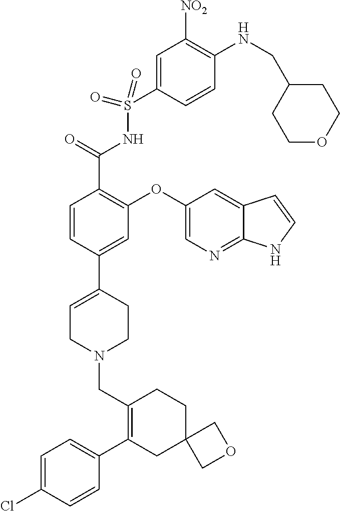 [SMILES: O=C(NS(=O)(=O)c1ccc(NCC2CCOCC2)c([N+](=O)[O-])c1)c1ccc(C2=CCN(CC3=C(c4ccc(Cl)cc4)CC4(CC3)COC4)CC2)cc1Oc1cnc2[nH]ccc2c1] | 2-((1H-pyrrolo[2,3-b]pyridin-5-yl)oxy)- 4-(1-((6-(4-chlorophenyl)-2- oxaspiro[3.5]non-6-en-7-yl)methyl)- 1,2,3,6-tetrahydropyridin-4-yl)-N-((3- nitro-4-(((tetrahydro-2H-pyran-4- yl)methyl)amino)phenyl)sulfonyl)benzamide |
| 7 | 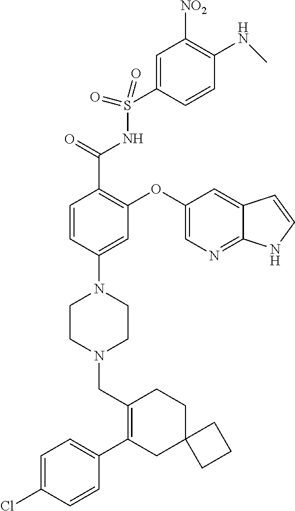 [SMILES: CNc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((4- (methylamino)-3- nitrophenyl)sulfonyl)benzamide |
| 8 | 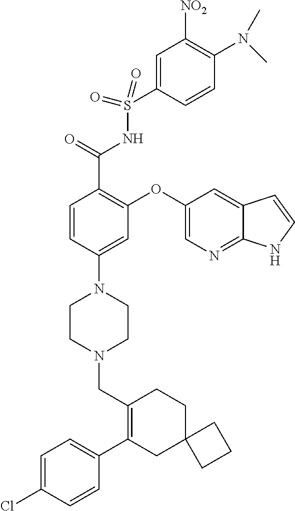 [SMILES: CN(C)c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((4- (dimethylamino)-3- nitrophenyl)sulfonyl)benzamide |
| 9 | 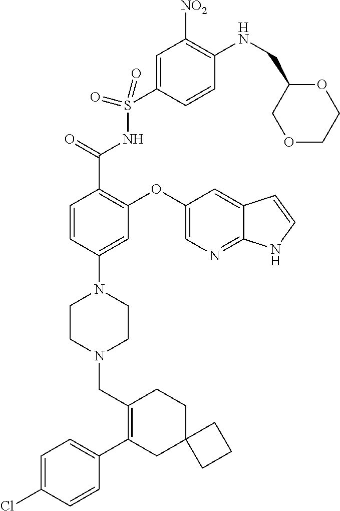 [SMILES: O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (R)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)benzamide |
| 10 | 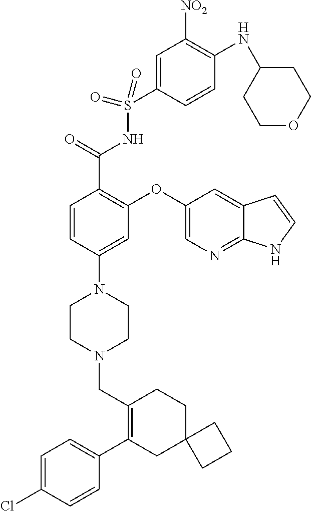 [SMILES: O=C(NS(=O)(=O)c1ccc(NC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-((tetrahydro-2H-pyran-4- yl)amino)phenyl)sulfonyl)benzamide |
| 11 | 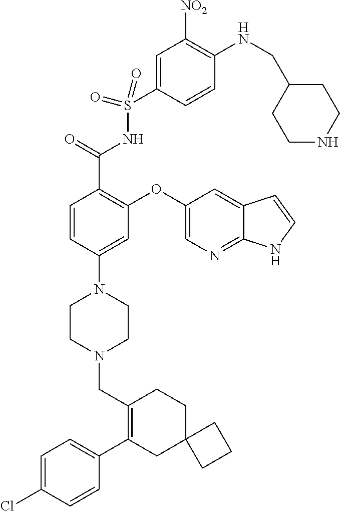 [SMILES: O=C(NS(=O)(=O)c1ccc(NCC2CCNCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-((piperidin-4- ylmethyl)amino)phenyl)sulfonyl)benzamide |
| 12 | 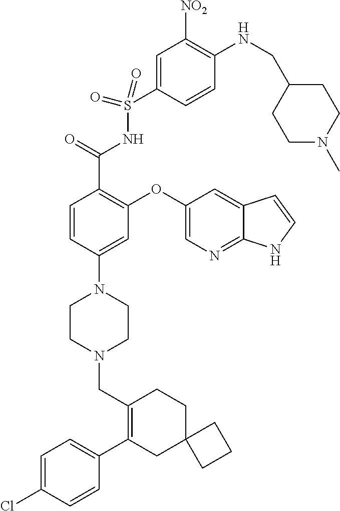 [SMILES: CN1CCC(CNc2ccc(S(=O)(=O)NC(=O)c3ccc(N4CCN(CC5=C(c6ccc(Cl)cc6)CC6(CCC6)CC5)CC4)cc3Oc3cnc4[nH]ccc4c3)cc2[N+](=O)[O-])CC1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((4- (((1-methylpiperidin-4- yl)methyl)amino)-3- nitrophenyl)sulfonyl)benzamide |
| 13 | 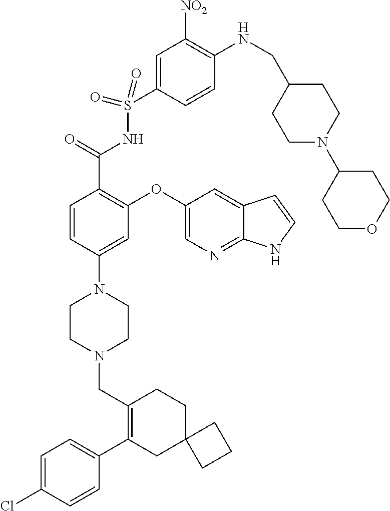 [SMILES: O=C(NS(=O)(=O)c1ccc(NCC2CCN(C3CCOCC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-(((1-(tetrahydro-2H-pyran-4- yl)piperidin-4- yl)methyl)amino)phenyl)sulfonyl)benzamide |
| 14 | 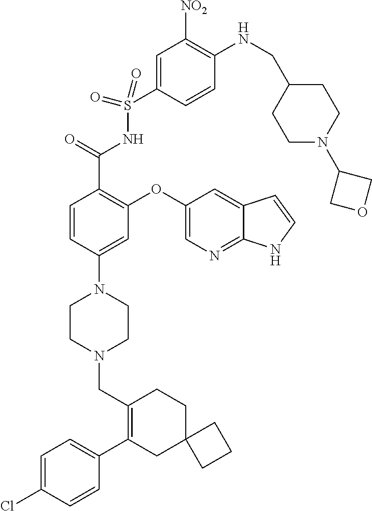 [SMILES: O=C(NS(=O)(=O)c1ccc(NCC2CCN(C3COC3)CC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-(((1-(oxetan-3-yl)piperidin-4- yl)methyl)amino)phenyl)sulfonyl)benzamide |
| 15 | 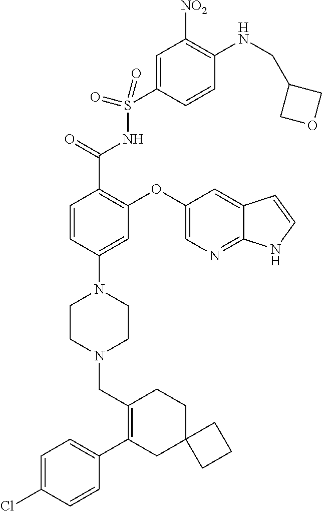 [SMILES: O=C(NS(=O)(=O)c1ccc(NCC2COC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- nitro-4-((oxetan-3- ylmethyl)amino)phenyl)sulfonyl)benzamide |
| 16 | 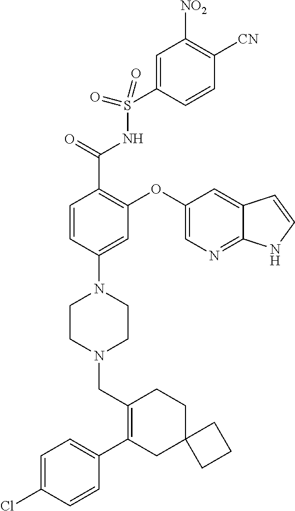 [SMILES: N#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((4- cyano-3-nitrophenyl)sulfonyl)benzamide |
| 17 | 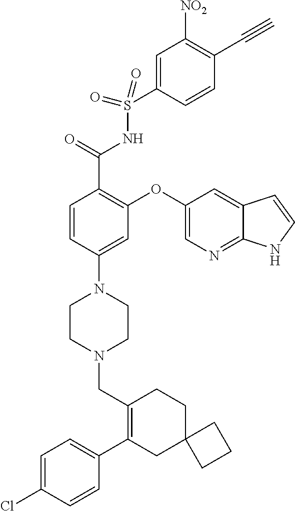 [SMILES: C#Cc1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((4- ethynyl-3- nitrophenyl)sulfonyl)benzamide |
| 18 | 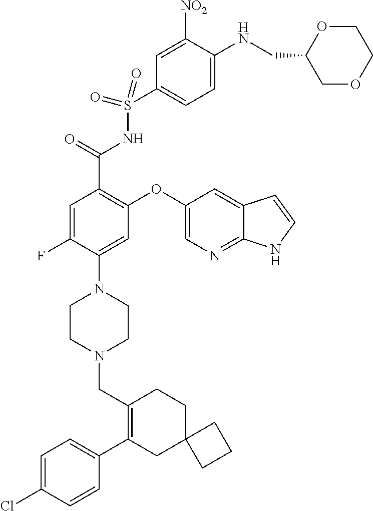 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)-5- fluorobenzamide |
| 19 | 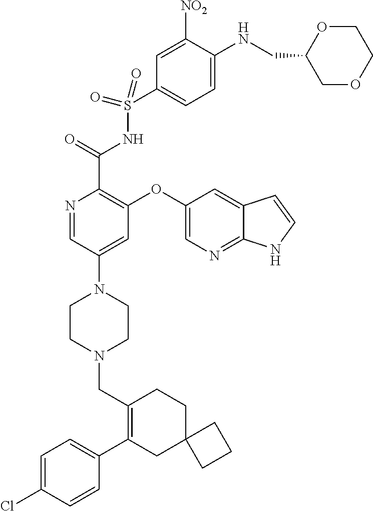 [SMILES: O=C(NS(=O)(=O)c1ccc(NC[C@H]2COCCO2)c([N+](=O)[O-])c1)c1ncc(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-3- nitrophenyl)sulfonyl)-3-((1H- pyrrolo[2,3-b]pyridin-5-yl)oxy)-5-(4-((6- (4-chlorophenyl)spiro[3.5]non-6-en-7- yl)methyl)piperazin-1-yl)picolinamide |
| 20 | 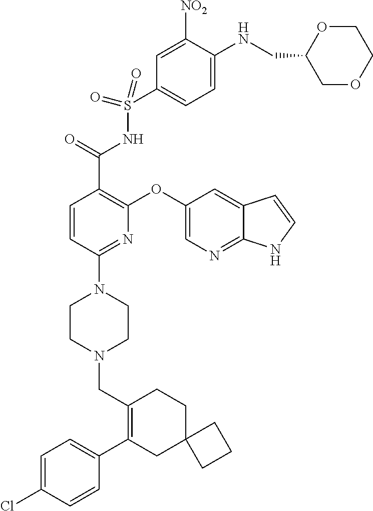 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)nc1Oc1cnc2[nH]ccc2c1] | (S)-N-((4-(((1,4-dioxan-2- yl)methyl)amino)-3- nitrophenyl)sulfonyl)-2-((1H- pyrrolo[2,3-b]pyridin-5-yl)oxy)-6-(4-((6- (4-chlorophenyl)spiro[3.5]non-6-en-7- yl)methyl)piperazin-1-yl)nicotinamide |
| 21 | 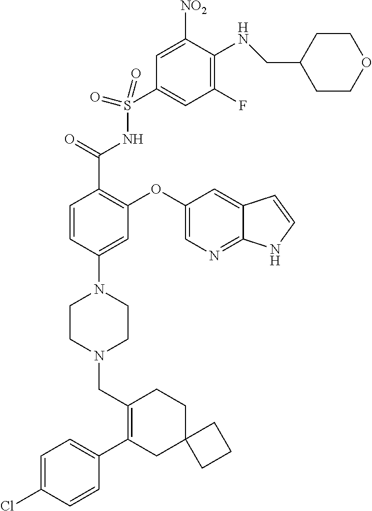 [SMILES: O=C(NS(=O)(=O)c1cc(F)c(NCC2CCOCC2)c([N+](=O)[O-])c1)c1ccc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-N-((3- fluoro-5-nitro-4-(((tetrahydro-2H-pyran- 4- yl)methyl)amino)phenyl)sulfonyl)benzamide |
| 22 | 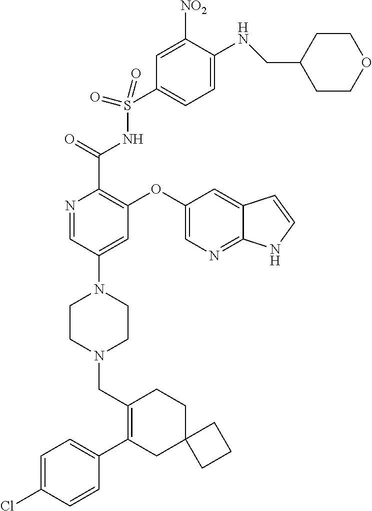 [SMILES: O=C(NS(=O)(=O)c1ccc(NCC2CCOCC2)c([N+](=O)[O-])c1)c1ncc(N2CCN(CC3=C(c4ccc(Cl)cc4)CC4(CCC4)CC3)CC2)cc1Oc1cnc2[nH]ccc2c1] | 3-((1H-pyrrolo[2,3-b]pyridin-5-yl)oxy)- 5-(4-((6-(4-chlorophenyl)spiro[3.5]non- 6-en-7-yl)methyl)piperazin-1-yl)-N-((3- nitro-4-(((tetrahydro-2H-pyran-4- yl)methyl)amino)phenyl)sulfonyl)picolinamide |
| 23 | 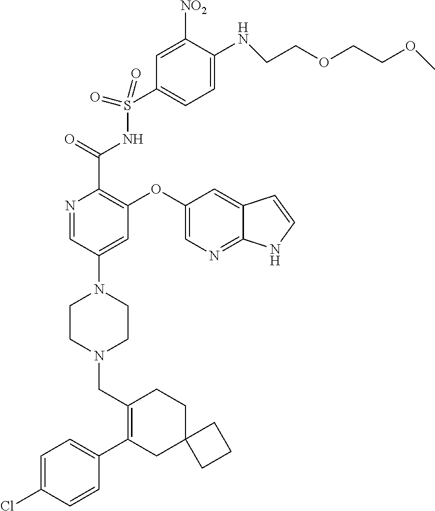 [SMILES: COCCOCCNc1ccc(S(=O)(=O)NC(=O)c2ncc(N3CCN(CC4=C(c5ccc(Cl)cc5)CC5(CCC5)CC4)CC3)cc2Oc2cnc3[nH]ccc3c2)cc1[N+](=O)[O-]] | 3-((1H-pyrrolo[2,3-b]pyridin-5-yl)oxy)- 5-(4-((6-(4-chlorophenyl)spiro[3.5]non- 6-en-7-yl)methyl)piperazin-1-yl)-N-((4- ((2-(2-methoxyethoxy)ethyl)amino)-3- nitrophenyl)sulfonyl)picolinamide |
| 24 | 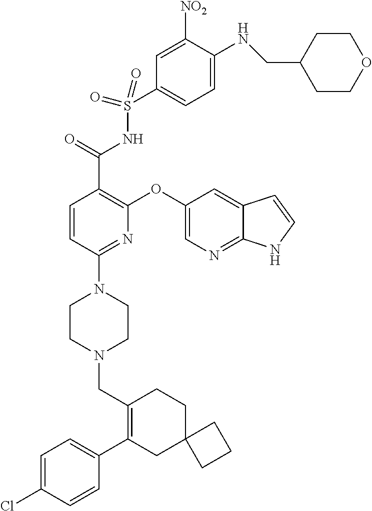 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)nc1Oc1cnc2[nH]ccc2c1] | 2-((1H-pyrrolo[2,3-b]pyridin-5-yl)oxy)- 6-(4-((6-(4-chlorophenyl)spiro[3.5]non- 6-en-7-yl)methyl)piperazin-1-yl)-N-((3- nitro-4-(((tetrahydro-2H-pyran-4- yl)methyl)amino)phenyl)sulfonyl)nicotinamide |
| 25 | 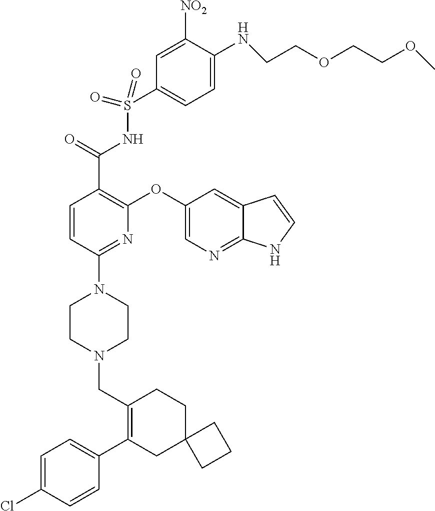 [SMILES: COCCOCCNc1ccc(S(=O)(=O)NC(=O)c2ccc(N3CCN(CC4=C(c5ccc(Cl)cc5)CC5(CCC5)CC4)CC3)nc2Oc2cnc3[nH]ccc3c2)cc1[N+](=O)[O-]] | 2-((1H-pyrrolo[2,3-b]pyridin-5-yl)oxy)- 6-(4-((6-(4-chlorophenyl)spiro[3.5]non- 6-en-7-yl)methyl)piperazin-1-yl)-N-((4- ((2-(2-methoxyethoxy)ethyl)amino)-3- nitrophenyl)sulfonyl)nicotinamide |
| 26 | 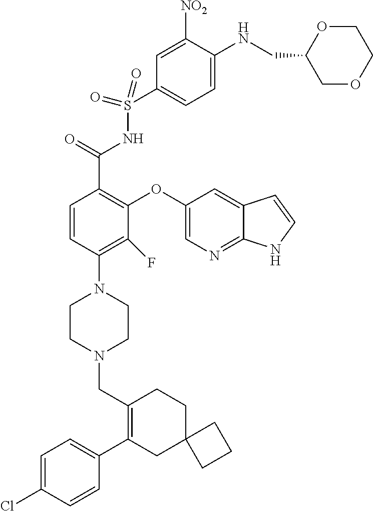 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | (S)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)-3- fluorobenzamide |
| 27 | 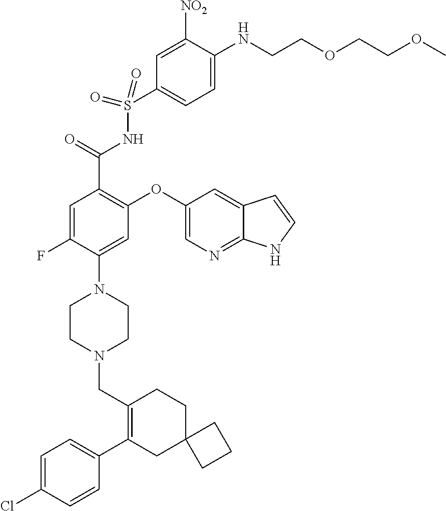 [SMILES: COCCOCCNc1ccc(S(=O)(=O)NC(=O)c2cc(F)c(N3CCN(CC4=C(c5ccc(Cl)cc5)CC5(CCC5)CC4)CC3)cc2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-5- fluoro-N-((4-((2-(2- methoxyethoxy)ethyl)amino)-3- nitrophenyl)sulfonyl)benzamide |
| 28 | 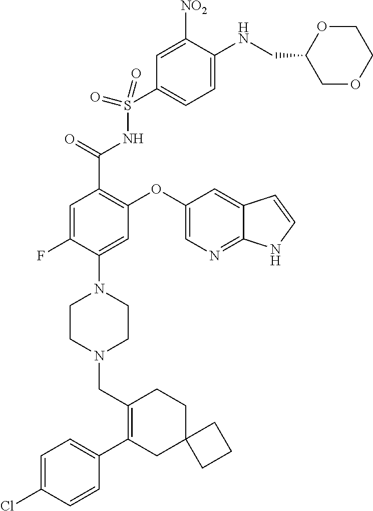 [SMILES: O=C(NS(=O)(=O)c1ccc(NC[C@H]2COCCO2)c([N+](=O)[O-])c1)c1cc(F)c(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)-5- fluorobenzamide |
| 29 | 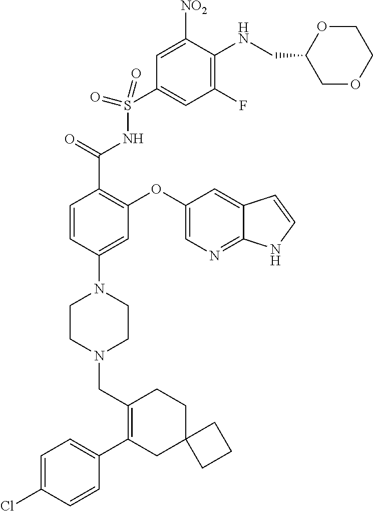 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-3-fluoro-5- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)benzamide |
| 30 | 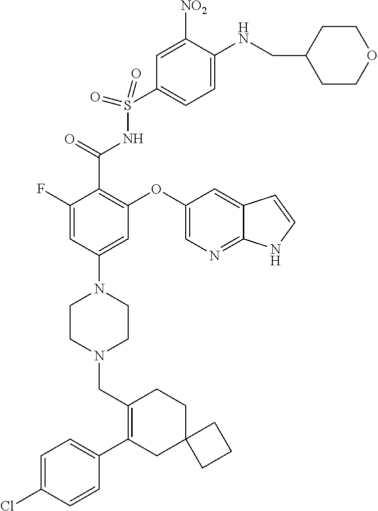 [SMILES: O=C(NS(=O)(=O)c1ccc(NCC2CCOCC2)c([N+](=O)[O-])c1)c1c(F)cc(N2CCN(CC3=C(c4ccc(Cl)cc4)CC4(CCC4)CC3)CC2)cc1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-6- fluoro-N-((3-nitro-4-(((tetrahydro-2H- pyran-4- yl)methyl)amino)phenyl)sulfonyl)benzamide |
| 31 | 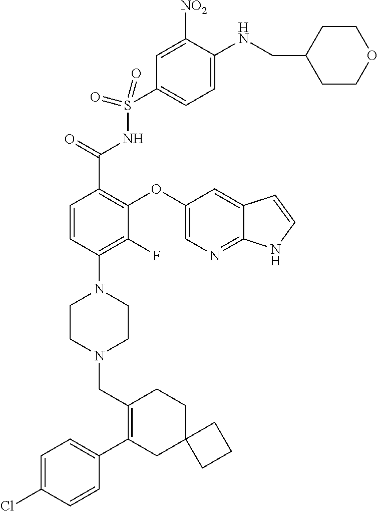 [SMILES: O=C(NS(=O)(=O)c1ccc(NCC2CCOCC2)c([N+](=O)[O-])c1)c1ccc(N2CCN(CC3=C(c4ccc(Cl)cc4)CC4(CCC4)CC3)CC2)c(F)c1Oc1cnc2[nH]ccc2c1] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-3- fluoro-N-((3-nitro-4-(((tetrahydro-2H- pyran-4- yl)methyl)amino)phenyl)sulfonyl)benzamide |
| 32 | 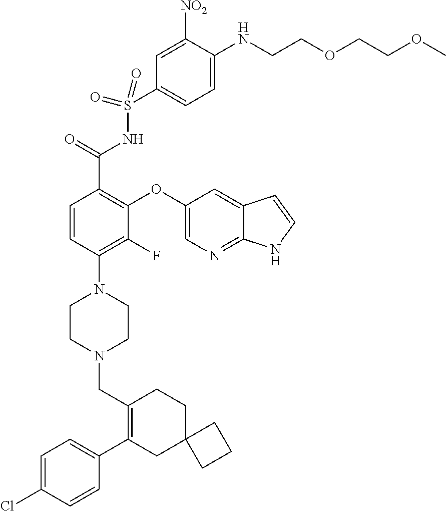 [SMILES: COCCOCCNc1ccc(S(=O)(=O)NC(=O)c2ccc(N3CCN(CC4=C(c5ccc(Cl)cc5)CC5(CCC5)CC4)CC3)c(F)c2Oc2cnc3[nH]ccc3c2)cc1[N+](=O)[O-]] | 2-((1H-pyrrolo[2,3-b]pyridin-5-yl)oxy)- 4-(4-((6-(4-chlorophenyl)spiro[3.5]non- 6-en-7-yl)methyl)piperazin-1-yl)-3- fluoro-N-((4-((2-(2- methoxyethoxy)ethyl)amino)-3- nitrophenyl)sulfonyl)benzamide |
| 33 | 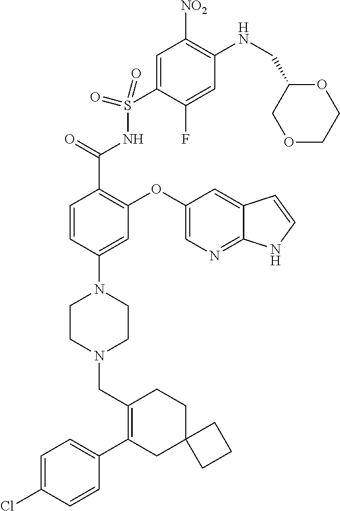 [SMILES: O=C(NS(=O)(=O)c1cc([N+](=O)[O-])c(NC[C@H]2COCCO2)cc1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-2-fluoro-5- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)benzamide |
| 34 | 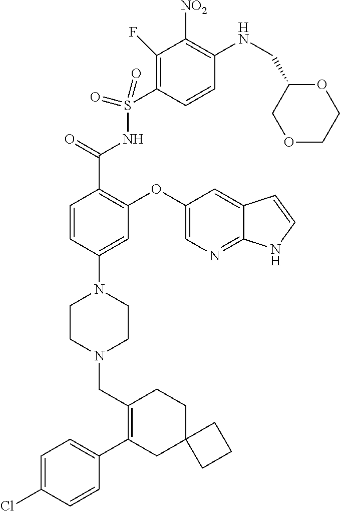 [SMILES: O=C(NS(=O)(=O)c1ccc(NC[C@H]2COCCO2)c([N+](=O)[O-])c1F)c1ccc(N2CCN(CC3=C(c4ccc(Cl)cc4)CC4(CCC4)CC3)CC2)cc1Oc1cnc2[nH]ccc2c1] | (S)-N-((4-(((1,4-dioxan-2- yl)methyl)amino)-2-fluoro-3- nitrophenyl)sulfonyl)-2-((1H- pyrrolo[2,3-b]pyridin-5-yl)oxy)-4-(4-((6- (4-chlorophenyl)spiro[3.5]non-6-en-7- yl)methyl)piperazin-1-yl)benzamide |
| 35 | 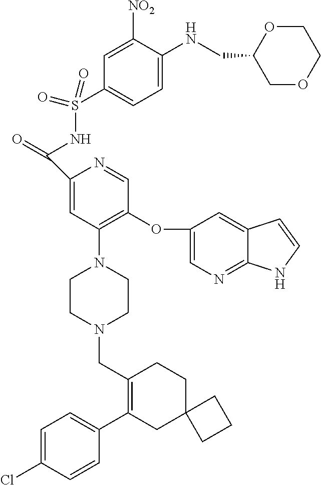 [SMILES: O=C(NS(=O)(=O)c1ccc(NC[C@H]2COCCO2)c([N+](=O)[O-])c1)c1cc(N2CCN(CC3=C(c4ccc(Cl)cc4)CC4(CCC4)CC3)CC2)c(Oc2cnc3[nH]ccc3c2)cn1] | (S)-N-((4-(((1,4-dioxan-2- yl)methyl)amino)-3- nitrophenyl)sulfonyl)-5-((1H- pyrrolo[2,3-b]pyridin-5-yl)oxy)-4-(4-((6- (4-chlorophenyl)spiro[3.5]non-6-en-7- yl)methyl)piperazin-1-yl)picolinamide |

In some embodiments, the compound of formula I is selected from the group consisting of:

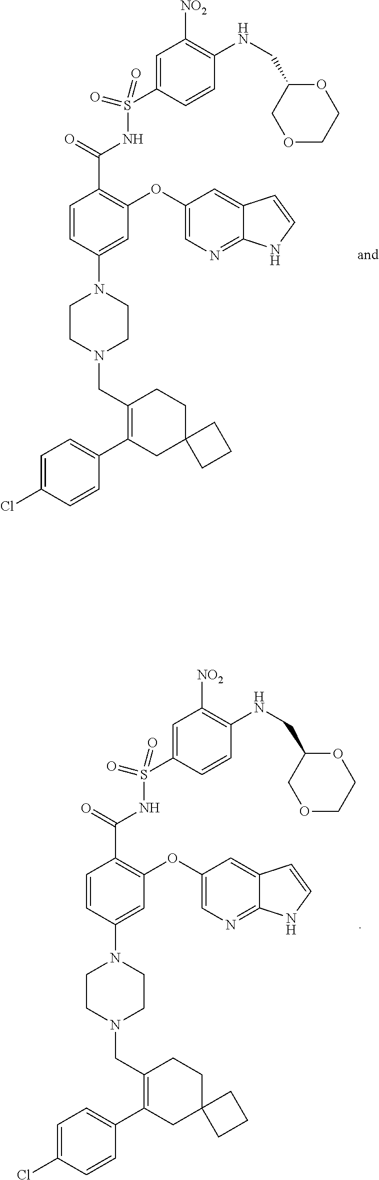 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the compound of formula I is selected from the group consisting of:

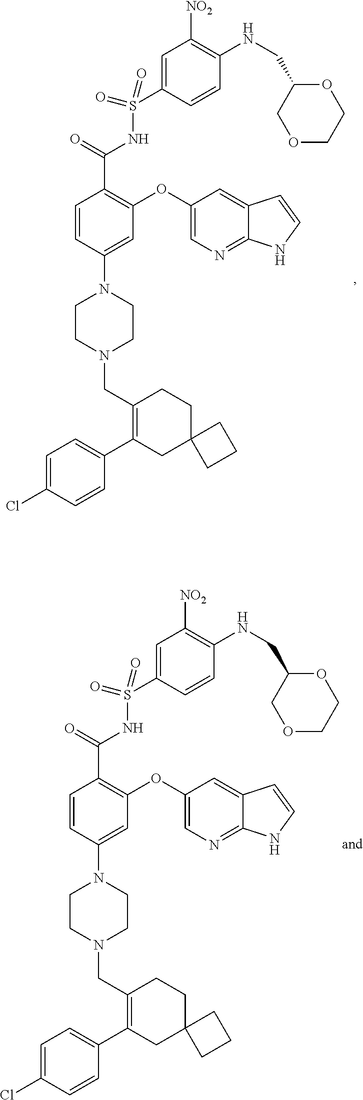 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

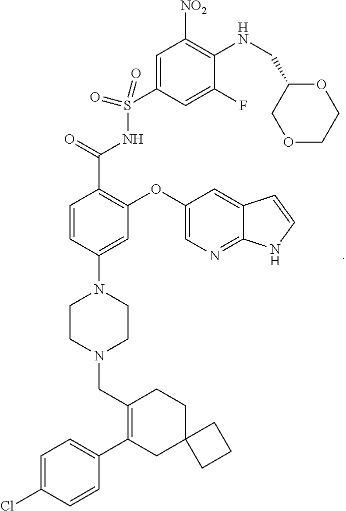 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the compound of formula I is:

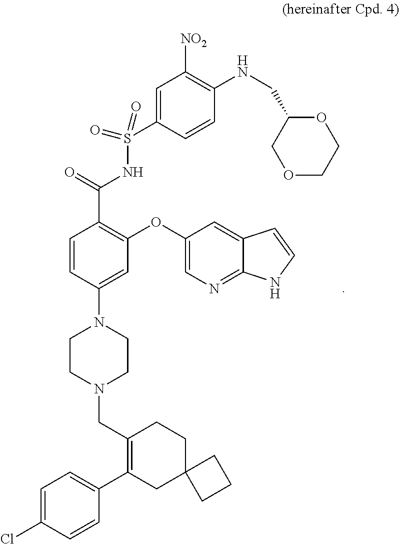 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the compound of formula I is:

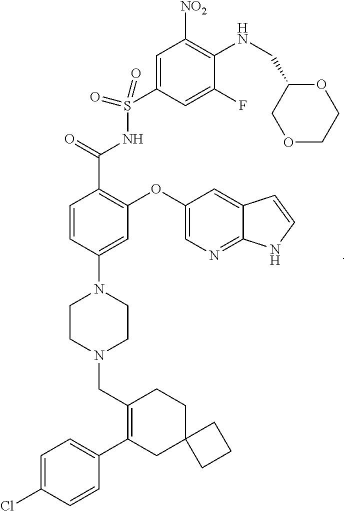 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

In some embodiments, the therapeutic agent is the compound of formula X, or a pharmaceutically acceptable salt or solvate thereof.

In some embodiments, the therapeutic agent is the compound of formula XI, or a pharmaceutically acceptable salt or solvate thereof. It has been known that Cpd. X is converted to Cpd. XI by metabolism in the body.

In some embodiments, the therapeutic agent (e.g., the compound of formula I) is administered orally. In some embodiments, the therapeutic agent (e.g., the compound of formula X) is administered by injection.

Without violating the common sense in the art, the above preferred conditions can be arbitrarily combined, then preferred embodiments of the disclosure are obtained.

All references described herein are incorporated by reference in their entireties.

The compounds described herein are known as BCL-2 inhibitor or BCL-2/BCL-xL dual inhibitor.

Inventors have found that BCL-2 inhibitor as well as BCL-2/BCL-xL dual inhibitor could inhibit innate immune, type 1, type 2, and type 3 immunities as evidenced by positive efficacy in the representative models, including DSS-induced acute IBD model, TNBS-induced acute IBD model, CD4⁺CD45RB^{high} T transfer IBD model, OVA-induced allergic asthma model, OXA (Oxazolone)-induced atopic dermatitis model, IMQ-induced psoriasis model, IL-23 induced psoriasis model, LPS inhalation induced lung neutrophils infiltration model and DTH model.

Mechanistical results confirmed that BCL-2 inhibitor or BCL-2/BCL-xL dual inhibitor could inhibit innate immune, type 1, type 2, and type 3 immunities. Detail results were showed as followings:
a) BCL-2 inhibitor and/or BCL-2/BCL-xL dual inhibitor inhibited the innate immune response by reducing elevated neutrophils, macrophages and/or natural killer cells in lymph nodes and/or decreasing the elevated blood neutrophils and/or blood monocytes, and/or decreasing the elevated neutrophils in lung;
b) BCL-2 inhibitor inhibited the type 1 immune response by reducing the activated Th1 cells, and/or elevated effector cells such as macrophages and NK cells in lymph nodes and/or monocytes in blood;
c) BCL-2 inhibitor or BCL-2/BCL-xL dual inhibitor inhibited type 2 immune response by reducing elevated lung eosinophils and/or serum IgE production in allergic mice model;
d) BCL-2 inhibitor inhibited the type 3 immune response by reducing the activated Th17 cells and/or elevated neutrophils in lymph node and/or elevated neutrophils in blood.

Given the above results, it is reasonable to assume that BCL-2 inhibitor Cpd. 4 as well as BCL/2 and BCL-xL dual inhibitor Cpd. X, CPD.XI may have a therapeutic role in immune diseases associated with innate immune, type 1, type 2, and type 3 immune responses. These diseases shall comprise, but not be limited to, the following items: acute disseminated encephalomyelitis (ADEM), Addison disease, ankylosing spondylitis, antiphospholipid syndrome (APGS), aplastic anemia, autoimmune haemolytic anaemia (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenic purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, Takayasu arthritis, Vogt-Kovangai-Harada disease, asthma, atopic dermatitis, allergic rhinitis, food allergy, acute urticaria, Contact dermatitis.

### EXAMPLES

The following examples further illustrate the present disclosure, but the present disclosure is not limited thereto.

### Example 1

### 1. Experimental Protocol for TNBS Induced Inflammatory Bowel Disease (IBD) Model in Balb/c Mice

There are two kinds of IBD: Crohn's disease (CD) and ulcerative colitis (UC). TNBS administration results in a preclinical mouse model replicating clinical Crohn's disease.

Female Balb/c mice (8 weeks old) were obtained from Beijing Vital River Laboratory Animal Co. Ltd. Animals were housed and handled in a temperature-controlled environment with a 12-h light/12-h dark cycle. A total of 50 mice were assigned to 5 groups by randomization based on body weight.

On Day 0, the mice weighing 18-20 g were anesthetized with Avidin (Easycheck, M2910). The mice were further intra-rectally injected 100 μL 1.5% TNBS solution (final concentration 50% ethanol) in the vehicle group and other treatment groups. For the Sham group, the mice were intra-rectally injected with 50% ethanol at the same volume.

Mice in Group 1 and Group 2 were treated with vehicle for 8 days (po, qd, from day −1 to day 6). Mice in Group 3 were treated with 50 mg/kg Cpd. 4 for 8 days (preventive regimen, po, qd, from day −1 to day 6). Mice in Group 4 were treated with 50 mg/kg Cpd. 4 for 6 days (therapeutic regimen, po, qd, from day 1 to day 6). 50 mg/kg Mesalamine was used as positive control which was orally administered to mice in Group 5 for 8 days (preventive regimen, qd, from day −1 to day 6). The protocols and procedures involving the care and use of animals were approved by the Institutional Animal Care and Use Committee (IACUC) at WuxiApptec (Shanghai, China). The grouping is shown in table 2.

TABLE 2

| | | | Number |
|---|---|---|---|
| Group | Treatment | Dosage | of mice |
| Group 1 (G1) | Sham | po, qd, from day −1 | 10 |
| | | to day 6 (8 D) | |
| Group 2 (G2) | Vehicle | po, qd, from day −1 | 10 |
| | | to day 6 (8 D) | |
| Group 3 (G3) | Cpd. 4 | 50 mg/kg, po, qd, from | 10 |
| | | day −1 to day 6 (8 D) | |
| Group 4 (G4) | Cpd. 4 | 50 mg/kg, po, qd, from | 10 |
| | | day 1 to day 6 (6 D) | |
| Group 5 (G5) | Mesalamine | 50 mg/kg, po, qd, from | 10 |
| | | day −1 to day 6 (8 D) | |

### 2. Clinical Scores

Clinical signs of IBD were assessed every day based on the score of disease activity index (DAI) which was evaluated from three parameters using a scoring system from 0 to 4: stool consistency (0, normal stool; 1, soft but still formed stool; 2, soft and not formed stool; 3, very soft and wet stool; 4, watery diarrhea), bleeding score (0, negative hemoccult; 1, weak positive hemoccult; 2, positive hemoccult; 3, blood trace in stool visible; 4, gross rectal bleeding) and body weight loss (0, no body weight loos; 1, 1-5% body weight loss; 2, 6-10% body weight loss; 3, 11-20% body weight loss; 4, >20% body weight loss).

TABLE 3

| DAI | BW | Stoll | |
|---|---|---|---|
| score | loss % | consistency | Bleeding |
| 0 | 0 | Normal | Negative hemoccult |
| 1 | 1-5 | Soft but | Weak positive |
| | | still formed | hemoccult |
| 2 | 6-10 | Soft | Positive hemoccult |
| 3 | 11-20 | Very soft; wet | Blood trace in stool |
| | | | visible |
| 4 | >20 | Watery diarrhea | Gross rectal bleeding |

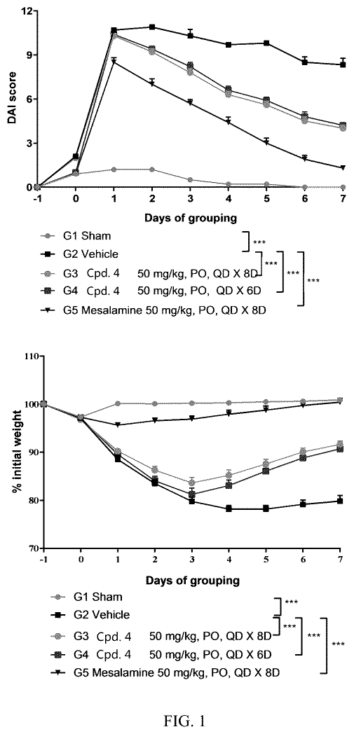

As seen in FIG. 1, mice from Group 1 (sham control) showed almost no symptoms of DAI (DAI score less than 2 during the entire study). Compared with G1 sham control group, after TNBS intrarectal instillation, there was a higher clinical score in the G2 group reaching the maximal clinical score of 10.70±0.14 on day 1. Treatment with mesalamine significantly lowered DAI during the entire experiment. Cpd. 4 showed efficacy by significantly reducing the clinical score of IBD and improving body weight loss when dosed at either of the two regimens. These results indicated that both preventive and therapeutic treatment of Cpd. 4 could ameliorate the progression TNBS-induced IBD in mice.

### 3. Colon Weight and Colon Length Measurement

At the end of induction with TNBS (day 7), the animals were euthanized, dissected and the entire colon was quickly removed and gently cleared of feces.

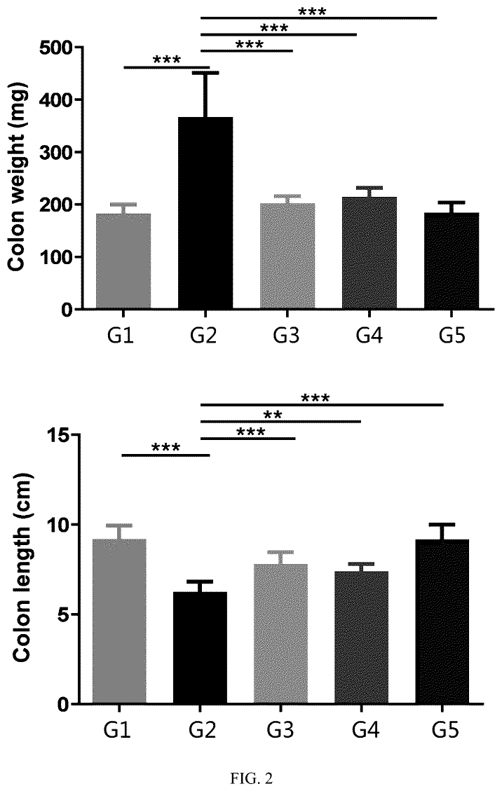

The entire colon was weighed, and the total length was measured. Colon weight gain and shortening is an indirect marker of inflammation. As expected, colon weight induced by TNBS increased to 336.8±25.15 mg. In consistency with the clinical score, treatment with mesalamine significantly reduced the colon weight. The TNBS-induced colon weight increasement were improved by Cpd. 4 treatment (202.4±3.95 mg for 50 mg/kg Cpd. 4 preventive therapy and 214.4±5.17 mg for 50 mg/kg Cpd. 4 therapeutic therapy) (FIG. 2).

Colon length induced by TNBS decreased to 6.26±0.16 cm. Treatment with mesalamine significantly increased the colon length. 50 mg/kg Cpd. 4 preventive treatment improved the colon length to 7.81±0.2 cm and 50 mg/kg Cpd. 4 therapeutic treatment improved the colon length to 7.39±0.12 cm (FIG. 2).

These results indirectly indicate that both preventive and therapeutic treatment of Cpd. 4 improved the inflammation in colon in TNBS-induced colitis mice.

### 4. Whole Blood Cell Counting

At the end of induction with TNBS, the animals were euthanized, and blood was collected immediately by the heart punctures and used for whole blood cell analysis.

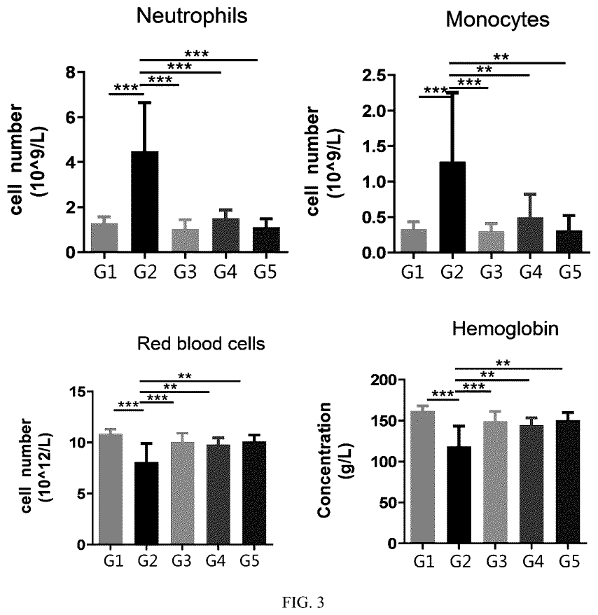

Neutrophils and macrophages, as the important components of the innate immune response, are key regulators of intestinal microenvironment homeostasis and to promote the development of IBD. Macrophages are derived from monocytes from peripheral blood. As shown in FIG. 3, TNBS induced models had significant increased neutrophils and monocytes in peripheral blood when compared with sham control. Both preventive and therapeutic treatments of Cpd. 4 significantly reduced the elevated cell number of neutrophils and monocytes in peripheral blood. Anemia is the most common extraintestinal manifestation of IBD. TNBS induction leaded to reduction of red blood cells and hemoglobin in peripheral blood, and treatment with Cpd. 4 improved anemia in TNBS induced mice IBD model.

### 5. Analysis of Cell Populations by Flow Cytometry

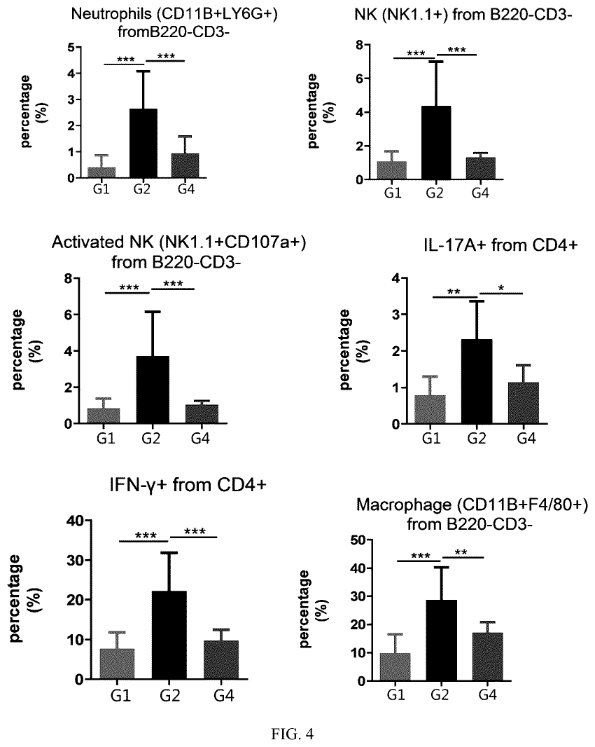

At the end of the study (day 7), the cell suspensions from sham control, model control and Cpd. 4 therapeutic treatment groups were obtained from mesenteric lymph nodes (MLN). Cells were stained with the following florescence-labelled antibodies: APC-Cy7-conjugated anti-mouse CD45, BV510-conjugated anti-mouse CD3e, AF700-conjugated anti-mouse CD8a, BUV395-conjugated anti-mouse CD4, BV421-conjugated anti-mouse CD25, FITC-conjugated anti-mouse Foxp3, BV650-conjugated anti-mouse IFN-γ, BV786-conjugated anti-mouse IL-17A, BB700-conjugated anti-mouse TNF-α, APC-conjugated anti-mouse B220, BV395-conjugated anti-mouse CD3e, BV605-conjugated anti-mouse CD11b, BB700-conjugated anti-mouse CD11c, AF488-conjugated anti-mouse MHCII, BV421-conjugated anti-mouse NK1.1, BV510-conjugated anti-mouse Ly6G, PE-Cy7-conjugated anti-mouse CD107a and PE-CF594-conjugated anti-mouse F4/80. All cells were primarily gated on single and live lymphocytes based on forward scatter (FCS), side scatter (SSC) and live/dead staining buffer. Samples were analyzed on a flow cytometer (BD LSRFortessa) to count the percentage of each subtype of lymphocytes. As seen in FIG. 4, after therapeutic treatment with 50 mg/kg Cpd. 4, the percentages of neutrophils (CD45+CD3−B220−CD11B+LY6G+), NK cells (CD45+CD3−B220−NK1.1+), activated NK cells (CD45+CD3−B220−NK1.1+CD107a+), IL-17A secreting CD4+ T cells (CD45+CD3+CD4+IL-17A+, Th17 cells), IFN-γ secreting CD4+T cells (CD45⁺CD3⁺CD4⁺IFN-γ⁺, Th1 cells), and macrophages (CD45+CD3−B220−CD11b+F4/80+) were significantly reduced when compared with vehicle control.

### 6. Histopathologic Assessment

At the end of the study (day 7), all the animals were sacrificed by CO₂. The colon was Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

As seen in FIG. 5, mice in the vehicle group (G2) showed a pathological score of 10.33±0.35, while Cpd. 4 preventive and therapeutic treatment significantly reduced the score to 2.70±0.80 and 5.00±1.15 respectively, which was in agreement with the alleviation of clinical symptoms as indicated by the clinical scores.

### 7. Masson's Trichrome Staining

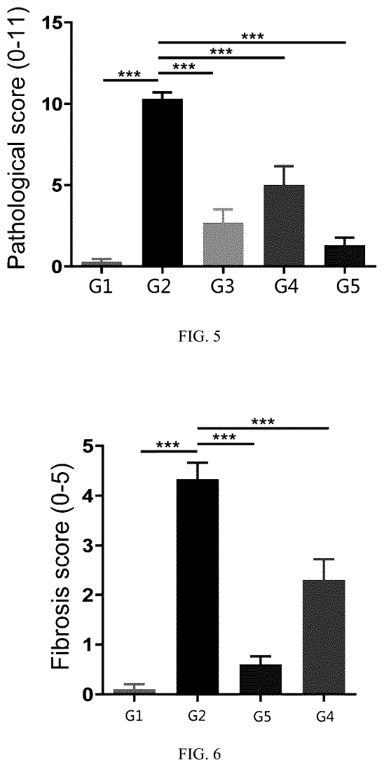

The fixed colon was stained with Masson's Trichrome to assess the collagen fibers in colon tissue. The Masson's Trichrome staining procedures was followed the standard protocol. After that, the pathological doctors from WuXi clinical pathological analysis platform reviewed the whole slices and scored blinded with animal information. The pathological scoring standards were as follows: No increase—0, Increased in the submucosa—1, Increased in the mucosa—2, Increased in the muscularis mucosa with thickening/disorganization of the muscularis mucosa—3, Increased in the muscularis propria (evident increases in collagen fibrils for Sirius red)—4, Gross disorganization of the muscularis propria—5. As seen in FIG. 6, Masson's Trichrome staining showed marked evidence of fibrosis in vehicle control group, compared to G1 sham control. Cpd. 4 therapeutic treatment significantly reduced the fibrosis score.

### Example 2

1. Experimental Protocol for CD4⁺CD45RB^{high} T Transfer Inflammatory Bowel Disease (IBD) Model in CB17 SCID Mice.

CD4⁺CD45RB^{high} T transfer model shows clinical and histological similarities to Crohn's Disease.

Balb/c mice and CB17 SCID were purchased from Zhejiang Vital River Laboratory Animal Technology Co., Ltd. (Zhejiang, China). Animals were housed and handled in a temperature-controlled environment with a 12-h light/12-h dark cycle. A total of 50 CB17 SCID mice were assigned to 5 groups by randomization based on body weight.

CD4⁺ T cells were isolated from the spleens of Balb/c mice using negative magnetic bead separation kit (Miltenyi Biotec) according to the manufacturers' instructions. The CD4⁺CD45RB^{high} T and CD4⁺CD45RB^{low} T cells were selected by flow cytometry and used for model construction. The isolated CD4⁺CD45RB^{high} T cells were intraperitoneally injected into 40 recipient CB17 SCID mice with 5×10⁵ cells per animal. Ten mice in Group 1 (G1) were intraperitoneally injected with 5×10⁵ CD4⁺CD45RB^{low} T cells as negative control group.

40 recipient CB17 SCID mice injected with CD4⁺CD45RB^{high} T cells were assigned to four groups (n=10 for each group): G2, vehicle control (po, qd, from day 0 to day 42); G3, 30 mg/kg Cpd. 4 (po, qd, from day 0 to day 42); G4, 30 mg/kg Cpd. 4 (po, qd, from day 14 to day 42); G5, 100 mg/kg Cpd. 4 (po, qd, from day 14 to day 42); G6, positive control, anti-mTNF-α 25 mg/kg (ip, Q3D, from day 14 to day 42).

The protocols and procedures involving the care and use of animals were approved by the Institutional Animal Care and Use Committee (IACUC) at Wuxi Apptec (Shanghai, China).

### 2. Clinical Scores

Clinical signs of IBD were assessed every 3 day based on the scores of disease activity index (DAI) which was evaluated from two parameters using a scoring system from 0 to 4: stool consistency (0, normal stool; 1, soft but still formed stool; 2, soft and not formed stool; 3, very soft and wet stool; 4, watery diarrhea) and body weight loss (0, no body weight loos; 1, 1-5% body weight loss; 2, 6-10% body weight loss; 3, 11-20% body weight loss; 4, >20% body weight loss).

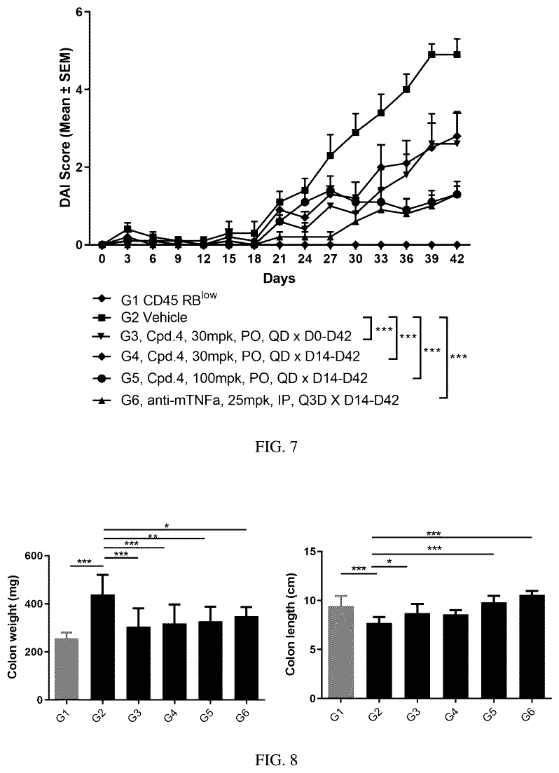

As seen in FIG. 7, mice from G1 (negative control) showed almost no symptoms of DAI (DAI score less than 1 during the entire study). Compared with G1, the DAI score grew gradually reaching the maximal clinical score of 4.9 at the end of the study in the G2 vehicle control. Treatment with anti-mTNF-α showed efficacy, with significantly lower DAI during the experiment. Cpd. 4 showed efficacy by significantly reducing the clinical score of IBD in G3, G4 and G5. These results indicate that treatment of Cpd. 4 ameliorated the progression in CD4⁺CD45RB^{high} T transfer mice IBD model.

### 3. Colon Weight and Colon Length Measurement

At the end of experiment, the animals were euthanized, dissected and the entire colon was quickly removed and gently cleared of feces.

The entire colon was weighed, and the total length was measured. As expected, colon weight increased significantly to 439.1 mg and colon length decreased to 7.72 cm in G2 CD4⁺CD45RB^{high} T transfer vehicle control when compared with G1 negative control. Treatment with anti-mTNF-α showed efficacy on both colon weight and length. Cpd. 4 significantly decreased colon weight in all treatment groups and significantly increased colon length in G3 and G5 (FIG. 8).

These results indirectly indicate that treatment of Cpd. 4 improved the inflammation in colon in CD4⁺CD45RB^{high} T transfer mice IBD model.

### 4. Histopathologic Assessment

The colons were Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

At the end of the study (day 42), all the animals were sacrificed by CO₂, and the colon tissues were collected and fixed in 10% formalin for pathological analysis (H&E and LFB staining). The histopathological score was evaluated microscopically in a blinded manner.

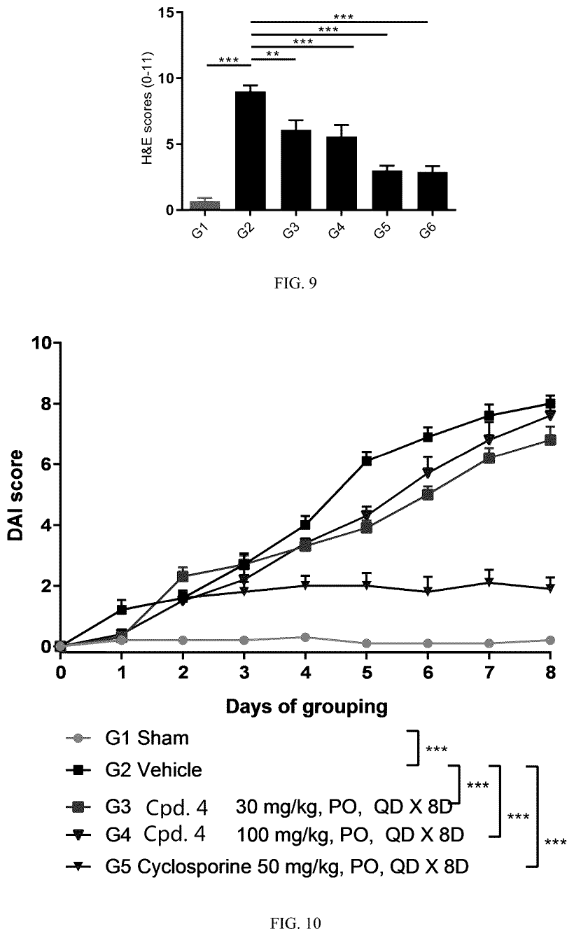

As seen in FIG. 9, compared with G1 negative control, mice in the vehicle group (G2) showed significant increase of pathological score, while Cpd. 4 treatment significantly reduced the score in all Cpd.4 groups, which was in agreement with the alleviation of clinical symptoms as indicated by the clinical scores.

### Example 3

### 1. Experimental Protocol for DSS Induced Inflammatory Bowel Disease (IBD) Model in C57BL/6 Mice

DSS-induced colitis shows clinical and histological similarities to ulcerative colitis.

Female C57BL/6 mice (8 weeks old) were obtained from Beijing Vital River Laboratory Animal Co. Ltd. Animals were housed and handled in a temperature-controlled environment with a 12-h light/12-h dark cycle. A total of 50 mice were assigned to 5 groups by randomization based on body weight.

On Day 0, colitis was induced by administration of 3% DSS (dextran sodium sulfate, molecular weight 36,000-50,000) in drinking water ad libitum for 8 days. 8-week-old mice were divided into 5 groups: Group 2, DSS treatment group (vehicle, po, qd, from day 0 to day 7); Group 3, DSS with 30 mg/kg Cpd. 4 (po, qd, from day 0 to day 7); Group 4, DSS with 100 mg/kg Cpd. 4 (po, qd, from day 0 to day 7); and Group 5, DSS with 50 mg/kg Cyclosporine (po, qd, from day 0 to day 7). For the Group 1 (G1) Sham group, the mice were provided drinking water without DSS.

TABLE 4

| | | | Number |
|---|---|---|---|
| Group | Treatment | Dosage | of mice |
| Group 1 (G1) | Sham | po, qd, from day 0 | 10 |
| | | to day 7 (8 D) | |
| Group 2 (G2) | Vehicle | po, qd, from day 0 | 10 |
| | | to day 7 (8 D) | |
| Group 3 (G3) | Cpd. 4 | 30 mg/kg, po, qd, from | 10 |
| | | day 0 to day 7 (8 D) | |
| Group 4 (G4) | Cpd. 4 | 100 mg/kg, po, qd, from | 10 |
| | | day 0 to day 7 (8 D) | |
| Group 5 (G5) | Cyclosporine | 50 mg/kg, po, qd, from | 10 |
| | | day 0 to day 7 (8 D) | |

The protocols and procedures involving the care and use of animals were approved by the Institutional Animal Care and Use Committee (IACUC) at Wuxi Apptec (Shanghai, China).

### 2. Clinical Scores

Clinical signs of IBD were assessed every day based on the scores of disease activity index (DAI) which was evaluated from three parameters using a scoring system from 0 to 4: stool consistency (0, normal stool; 1, soft but still formed stool; 2, soft and not formed stool; 3, very soft and wet stool; 4, watery diarrhea), bleeding score (0, negative hemoccult; 1, weak positive hemoccult; 2, positive hemoccult; 3, blood trace in stool visible; 4, gross rectal bleeding) and body weight loss (0, no body weight loos; 1, 1-5% body weight loss; 2, 6-10% body weight loss; 3, 11-20% body weight loss; 4, >20% body weight loss).

TABLE 5

| DAI | BW | Stoll | |
|---|---|---|---|
| score | loss % | consistency | Bleeding |
| 0 | 0 | Normal | Negative hemoccult |
| 1 | 1-5 | Soft but | Weak positive |
| | | still formed | hemoccult |
| 2 | 6-10 | Soft | Positive hemoccult |
| 3 | 11-20 | Very soft; wet | Blood trace in stool |
| | | | visible |
| 4 | >20 | Watery diarrhea | Gross rectal bleeding |

As seen in FIG. 10, mice from Group 1 (sham control) showed almost no symptoms of DAI (DAI score less than 1 during the entire study). Compared with G1 sham control group, in the Group 2 (DSS model control), the DAI score grew gradually reaching the maximal clinical score of 8.80±0.24 at the end of the study. Treatment with cyclosporine showed efficacy, with significantly lower DAI during the entire experiment. Cpd. 4 showed efficacy by significantly reducing the clinical score of IBD when dosed at either of the two doses. These results indicate that treatment of Cpd. 4 ameliorated the progression DSS-induced colitis in mice.

### 3. Colon Weight and Colon Length Measurement

At the end of induction with DSS, the animals were euthanized, dissected and the entire colon was quickly removed and gently cleared of feces.

The entire colon was weighed, and the total length was measured. Colon weight gain and shortening is an indirect marker of inflammation. As expected, DSS increased colon weight to 243.8±10.73 mg. Cpd. 4 significantly decreased colon weight, especially the colon weight increasement was significantly improved to 188.4±10.48 mg by 30 mg/kg Cpd. 4 treatment (FIG. 11).

For the colon length, DSS decreased the colon length to 4.81±0.08 cm. Cpd. 4 significantly increased length, especially treatment with 30 mg/kg Cpd. 4 significantly improved the colon length to 5.25±0.11 cm (FIG. 11).

These results indirectly indicate that treatment of Cpd. 4 improved the inflammation in colon in DSS-induced colitis mice.

### 4. Histopathologic Assessment

The colon of a mouse was Swiss-rolled and fixed with neutralized PFA followed by H&E staining. After that, the pathologists from WuXi clinical pathological analysis platform, who were blinded to animal ID, reviewed the H&E staining and scored. The pathological scoring standards were as follows: crypt architecture (normal, 0; severe crypt distortion with loss of entire crypts, 3), degree of inflammatory cell infiltration (normal, 0; dense inflammatory infiltrate, 3), muscle thickening (normal, 0; marked muscle thickening present, 3), goblet cell depletion (absent, 0; present, 1) and crypt abscess (absent, 0; present, 1).

At the end of the study (day 8), all the animals were sacrificed by CO2, and the colon tissues were collected and fixed in 10% formalin for pathological analysis (H&E and LFB staining). The histopathological score was evaluated microscopically in a blinded manner.

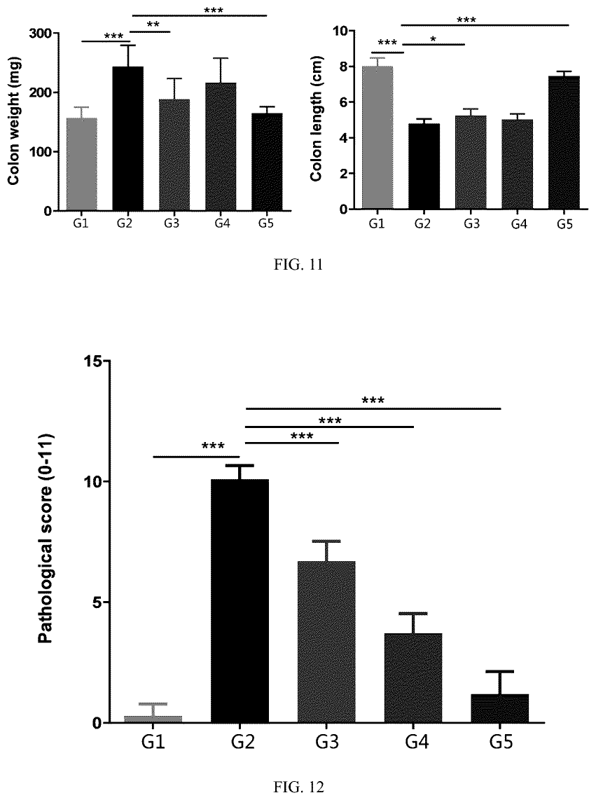

As seen in FIG. 12, mice in the vehicle group (G2) showed a pathological score of 10.10±0.17, while Cpd. 4 treatment dose-dependently significantly reduced the score to 6.70±0.25 (30 mg/kg) and 3.70±0.25 (100 mg/kg), which was in agreement with the alleviation of clinical symptoms as indicated by the clinical scores.

### Example 4

### 1. Experimental Protocol for OVA (Ovalbumin) Induced Asthma Model in Balb/cA Mice

Ovalbumin (OVA)-sensitized and challenged BALB/c mice are widely used as an asthma model and are characterized by high levels of serum IgE, airway inflammation, epithelial hypertrophy, goblet cell hyperplasia, and AHR, which are similar to the features observed in human allergic asthma.

Female Balb/cA mice (8 weeks old) were obtained from Shanghai Jihui Laboratory Animal Co. Ltd. Animals were housed and handled in a temperature-controlled environment with a 12-h light/12-h dark cycle. A total of 66 mice were assigned to 6 groups by randomization based on body weight.

Mice in Group 2 are intraperitoneally immunized with OVA (25 μg) emulsified in Imject alum (2.0 mg of Al[OH]₃) on day 1. During this sensitization phase, the mice produce anti-OVA IgE antibodies, which bind IgE receptors on mast cells. After this sensitization, the mice are intratracheally challenged with OVA from day 8 today 14, resulting in OVA cross-linked IgE on mast cells and leading to degranulating mast cells. Mice then develop clinical features of asthma. The mice in the Group 1 normal control group were sensitized and challenged with 0.09% saline (without OVA).

Mice in Group 1 and Group 2 were treated with vehicle for 7 days (po, qd, from day 8 to day 14). Mice in Group 3 were treated with 50 mg/kg Cpd. X for 8 days (iv, biw, day 1 and day 4). Mice in Group 4 were treated with 30 mg/kg Cpd. 4 for 7 days (po, qd, from day 8 to day 14). Mice in Group 5 were treated with 100 mg/kg Cpd. 4 for 7 days (po, qd, from day 8 to day 14). 3 mg/kg dexamethasone was used as positive control which was orally administered to mice in Group 6 for 7 days (qd, from day 8 to day 14). The protocols and procedures involving the care and use of animals were approved by the Institutional Animal Care and Use Committee (IACUC) at KCI (Suzhou, China). The grouping is shown in table 6.

TABLE 6

| | | | Number |
|---|---|---|---|
| Group | Treatment | Dosage | of mice |
| Group 1 (G1) | Sham | po, qd, from day 8 | 11 |
| | | to day 14 (7 D) | |
| Group 2 (G2) | Vehicle | po, qd, from day 8 | 11 |
| | | to day 14 (7 D) | |
| Group 3 (G3) | Cpd. X | 50 mg/kg, iv, biw, | 11 |
| | | day 1 and day 4 | |
| Group 4 (G4) | Cpd. 4 | 30 mg/kg, po, qd, from | 11 |
| | | day 8 to day 14 (7 D) | |
| Group 5 (G5) | Cpd. 4 | 100 mg/kg, po, qd, from | 11 |
| | | day 8 to day 14 (7 D) | |
| Group 6 (G6) | Dexamethasone | 3 mg/kg, po, qd, from | 11 |
| | | day 8 to day 14 (7 D) | |

### 2. Measurement of Airway Hyper-Responsiveness

Airway responsiveness was determined invasively based on lung resistance.

After challenge with aerosolized methacholine. Briefly, mice were challenged with methacholine aerosol in increasing concentrations (0, 6.25, 12.5, and 25 mg/mL in saline) on day 13 by using the noninvasive pulmonary function instrument. Data on lung resistance were continuously collected, and mean values were selected to express changes in airway function and regarded as one form of inflammation expression. Data are presented as the airway stenosis index (penh).

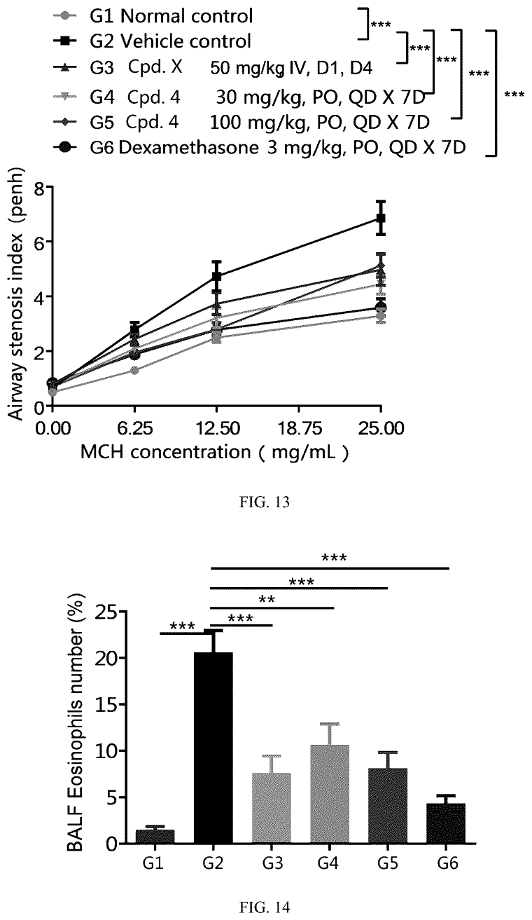

As seen in FIG. 13, following the sensitization and challenge protocol, a well-established animal model of OVA-induced asthma was established indicated by high airway stenosis index to calculate the effect of BCL-2 inhibitor Cpd. 4 and Cpd. X on asthmatic mice. In addition, in a Mch dose-dependent manner, BCL-2 inhibitor Cpd. 4 and Cpd. X administration showed significant inhibitory activity in the AHR of mice. Treatment with dexamethasone showed efficacy, with lower airway stenosis index during the entire experiment.

The data above suggested that BCL-2 inhibitor Cpd. 4 and Cpd. X had a potential role in the regulation of AHR progression.

### 3. BAL Fluid Cell Counts

Twenty-four hours after the final OVA challenge (on day 15), mice were killed, and BAL cells were collected by means of slow injection of ice-cold PBS into the trachea, which was repeated 3 times. Numbers of cells in BAL fluid were counted with a chamber. Number of eosinophils in a total of 100 cells was counted under a microscope. Results are expressed as percentage of eosinophils of BAL fluid.

Inflammatory response mediated by eosinophils is a major cause contributing to OVA-sensitization and challenge-induced airway inflammation. As shown in FIG. 14, BALF from the OVA model increased the percentage of eosinophils compared with sham control and dexamethasone significantly reduced the eosinophils as expected. The upregulated eosinophils in the BALF of OVA-induced mice were downregulated markedly by Cpd. 4 and Cpd. X treatment.

These results further indicated that Cpd. 4 and Cpd. X treatment are useful in airway inflammation via inflammation suppression.

### 4. IgE Production

Twenty-four hours after the final OVA challenge (on day 15), mice were killed, and the serum was collected. The OVA-specific IgE levels were measured with the method of enzyme linked immunosorbent assay (ELISA) according to manufacturer protocol.

Allergic asthma is characterized by overproduction of high level of serum IgE. As seen in FIG. 15, following sensitization and challenges, serum OVA-specific IgE in Group 2 were markedly increased compared with those of the G1 control group. The administration of Cpd. 4 and Cpd. X significantly reduced the levels of serum OVA-specific IgE while dexamethasone treatment reduced IgE in the OVA-challenged mice, too.

### 5. Histopathologic Assessment

Twenty-four hours after the final OVA challenge (on day 15), mice were killed, and the lung tissue samples were collected and fixed in 4% paraformaldehyde. Then, they were embedded in paraffin and cut into 4 μM sections for histopathological analysis. Lung sections were then stained with hematoxylin and eosin (H&E) to calculate the inflammatory changes. The histopathological score was evaluated microscopically in a blinded manner. Five arbitrarily selected fields of each mouse were photographed with an optical microscope and the images were determined and analyzed in detail. Histopathological evaluation was based on the intensity of the inflammatory infiltration and tissue injury in terminal bronchiole and pulmonary small arteries in randomly selected areas around the tissues and was scored as 0 (Normal structure with no inflammatory cell infiltration), 1 (a few scattered inflammatory cell infiltration (less than 10) but no focal), 2 (a lot scattered inflammatory cell infiltration which is focal or diffuse and totaled less than ½ area of the terminal bronchiole or the artery wall), 3 (diffuse infiltration of inflammatory cells and totaled more than ½ area of the terminal bronchiole or the pulmonary small artery wall, inflammatory cells infiltration in the medium layer of the membrane).

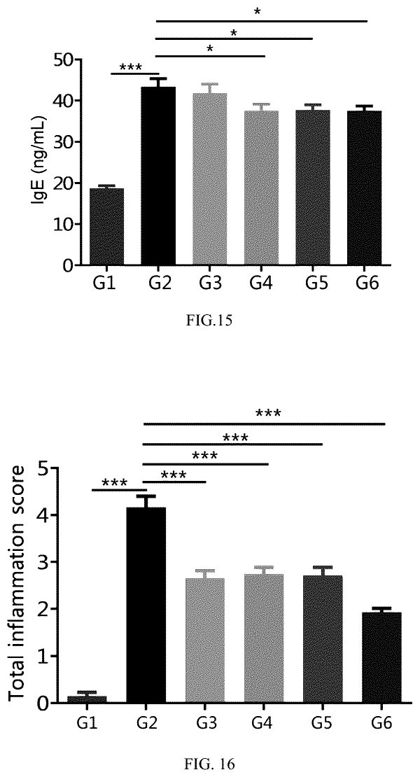

As seen in FIG. 16, mice in the vehicle group (G2) showed a pathological score of 4.08±0.23, while Cpd. 4 treatment significantly reduced the score to 2.64±0.08 (30 mg/kg) and 2.66±0.17 (100 mg/kg). Cpd. X treatment reduced the pathological score to 2.58±0.15.

The data above suggested that Cpd. 4 and Cpd. X ameliorated airway inflammation induced by OVA-sensitization and challenge in mice.

### Example 5

### 1. Experimental Protocol for OXA Induced Atopic Dermatitis (AD) Model in C57BL/6 Mice

Atopic dermatitis (AD) is a chronic skin disease that presents with itching, erythema and squamous lesions. Haptenating agent oxazolone (OXA) sensitization and continuous challenge establish AD like skin inflammation.

Female C57BL/6 mice (6 weeks old) were obtained from Biocytogen Laboratory Animal Co. Ltd. Animals were housed and handled in a temperature-controlled environment with a 12-h light/12-h dark cycle. A total of 48 mice were assigned to 6 groups by randomization based on body weight.

To induce AD-like lesion, all right ears of mice except the G1 sham control group were applied with oxazolone (OXA). Briefly, skin inflammation was induced by topical administration with 25 μL of 0.8% OXA dissolved in acetone on day 0, and repeated 25 μl of 0.4% OXA three times a week from day 7 to 25. G1 sham control group was treated same volume of vehicles. Therapeutic groups as 10 mg/kg, 30 mg/kg and 100 mg/kg Cpd. 4 (po, qd, day 7 to day 25) or 25 μL of 0.09% (w/v) dexamethasone (topical, qd, day 7 to day 25) were applied to the mice. The grouping is shown in table 7. The protocols and procedures involving the care and use of animals were approved by the Institutional Animal Care and Use Committee (IACUC) at Biocytogen (Beijing, China).

TABLE 7

| | | | Number |
|---|---|---|---|
| Group | Treatment | Dosage | of mice |
| Group 1 (G1) | Sham | po, qd, from day 7 | 8 |
| | | to day 25 (19 D) | |
| Group 2 (G2) | Vehicle | po, qd, from day 7 | 8 |
| | | to day 25 (19 D) | |
| Group 3 (G3) | Dexamethasone | 25 μL of 0.09% (w/v) | 8 |
| | | dexamethasone, topical, qd, | |
| | | from day 7 to day 25 (19 D) | |
| Group 4 (G4) | Cpd. 4 | 10 mg/kg, po, qd, from | 8 |
| | | day 7 to day 25 (19 D) | |
| Group 5 (G5) | Cpd. 4 | 30 mg/kg, po, qd, from | 8 |
| | | day 7 to day 25 (19 D) | |
| Group 6 (G6) | Cpd. 4 | 100 mg/kg, po, qd, from | 8 |
| | | day 7 to day 25 (19 D) | |

### 2. Ear Thickness Measurement

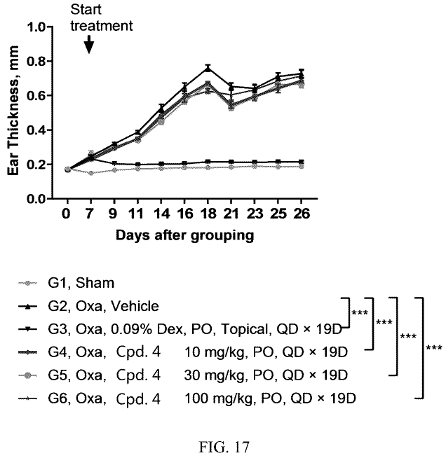

Ear thickness of mice was measured before application of Oxa and every other day from day 7 to day 26. As seen in FIG. 17, ear thickness of mice from Group 1 (sham control) maintained at 0.2 mm during the entire experiment. Compared with G1 sham control group, after OXA sensitization and challenge, ear swelling was observed indicated by increased ear thickness in G2 group reaching the maximal ear thickness to 0.76±0.02 mm on day 18. Treatment with dexamethasone showed superior efficacy, with significantly lower ear thickness during the entire experiment. Cpd. 4 showed efficacy in this mouse model of atopic dermatitis disease by significantly reducing the ear thickness when dosed at either of the three doses.

### Example 6

### 1. Experimental Protocol for IMQ Induced Psoriasis Model in Balb/c Mice

Imiquimod (IMQ) is a ligand for TLRs (Toll-like receptors) of immune cells (including macrophages, monocytes and plasmacytoid dendritic cells), and therefore contributes to strong activation of the immune system. The IMQ-induced Psoriasis model is translational into the clinic as it exhibits markers of human disease including histopathology and activation of the immune system, with a strong T-cell element.

Female Balb/c mice (7-8 weeks old) were obtained from B&K Universal Animal Co. Ltd. Animals were housed and handled in a temperature-controlled environment with a 12-h light/12-h dark cycle. A total of 36 mice were assigned to 4 groups by randomization based on body weight (6 mice in normal control group and 10 mice in other 3 groups).

At the beginning of the study on day 0, five groups were administered a daily topical dose of 80 mg of a cream preparation containing 5% IMQ on their hair-free backs to establish a model of IMQ-induced psoriasis for seven consecutive days. The G1 normal control group received appropriate vaseline.

Mice in Group 1 and Group 2 were treated with vehicle for 7 days (po, qd, from day 0 to day 6). Mice in Group 3 were treated with 100 mg/kg Cpd. 4 for 7 days (po, qd, from day 0 to day 6). Mice in Group 4 were treated with 30 mg/kg tofacitinib for 7 days (po, bid, from day 0 to day 6). The grouping is shown in table 8. The protocols and procedures involving the care and use of animals were approved by the Institutional Animal Care and Use Committee (IACUC) at KCI (Suzhou, China).

TABLE 8

| | | | Number |
|---|---|---|---|
| Group | Treatment | Dosage | of mice |
| Group 1 (G1) | Normal control | po, qd, from day 0 | 6 |
| | | to day 6 (7 D) | |
| Group 2 (G2) | Vehicle | po, qd, from day 0 | 10 |
| | | to day 6 (7 D) | |
| Group 3 (G3) | Cpd. 4 | 100 mg/kg, po, qd, from | 10 |
| | | day 0 to day 6 (7 D) | |
| Group 4 (G4) | Tofacitinib | 30 mg/kg, po, | 10 |

### 2. Severity Scoring of Skin Inflammation

The severity of skin inflammation was monitored and graded using a modified human scoring system Psoriasis Area Severity Index (PASI). Scaling, thickness, and erythema were scored separately on a scale from 0 to 4:0, none; 1, slight; 2, moderate; 3, marked; and 4, very marked. The total score denotes severity of inflammation. Ear thickness of mice was measured every day from day 0 to day 7.

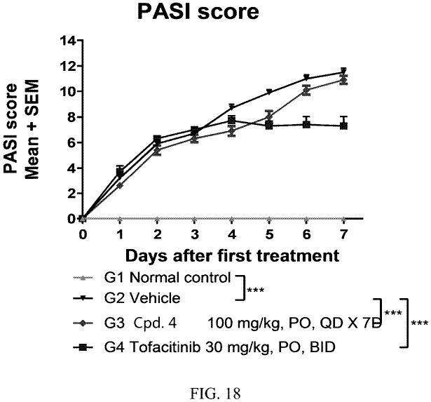

As seen in FIG. 18, mice from Group 1 (normal control) showed almost no symptoms of PASI. Compared with G1 normal control group, after IMQ treatment, the PASI score grew gradually in the G2 group reaching the maximal clinical score of 11.50±0.29 on day 7. Treatment with tofacitinib showed superior efficacy, with significantly lower PASI. It should be noted that the dose for tofacitinib was around 17-fold higher than its clinically relevant dose, plus obvious adverse effect observed in its clinical application. Cpd. 4 showed efficacy in this psoriasis mouse model by significantly reducing the clinical PASI score.

### 3. Ear Thickness Measurement

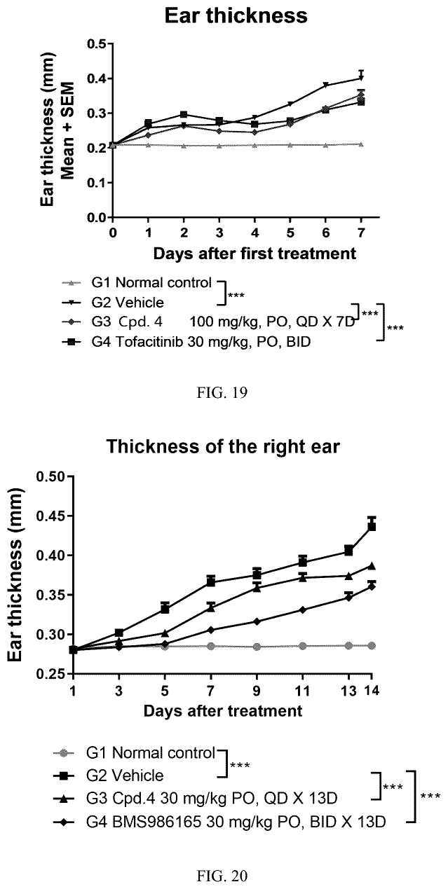

Ear thickness of mice was measured every day from day 0 to day 7. As seen in FIG. 19, ear thickness of mice from Group 1 (normal control) maintained at 0.2 mm during the entire experiment. Compared with G1 normal control group, after IMQ treatment, ear swelling was observed indicated by increased ear thickness in G2 group reaching the maximal ear thickness to 0.40±0.02 mm on day 7. Cpd. 4 showed efficacy in this mouse model of psoriasis by significantly reducing the ear thickness.

In addition, the efficacy of Cpd. 4 in reducing ear thickness was comparable to that of tofacitinib, the dose in mice model of which is 17-fold higher than its clinically relevant dose.

These results indicate that treatment of Cpd. 4 ameliorated the inflammation degree of IMQ-induced psoriasis in mice.

### Example 7

### 1. Experimental Protocol for IL-23 Induced Psoriasis Model in C57BL/6 Mice

IL-23 stimulates and promotes differentiation of Th17 cells. IL-23 is a heterodimeric cytokine with two subunits. It drives the Th17 response by its binding and signaling through its receptor subunits. When the IL-23R is activated, it promotes the development of Th17 cells and the resulting production of cytokines such as IL-17A, IL-17F, and IL-22, all which are involved in mediating psoriasiform changes.

C57BL/6 mice (7-8 weeks old) were obtained from Vital River Laboratory Animal Technology Co., Ltd. Animals were housed and handled in a temperature-controlled environment with a 12-h light/12-h dark cycle. A total of 40 mice were assigned to 4 groups by randomization based on body weight.

The first day of IL-23 injection is considered as Day 1. Intradermal injection of 20 μL PBS containing 500 ng recombinant mouse IL-23 into the right ear of anesthetized mice using a 30-gauge needle every other day for 14 days (Day 1, Day 3, Day 5, Day 7, Day 9, Day 11, Day 13), 7 times in total. The G1 normal control group intradermal injected with 20 μL PBS.

Mice in Group 1 and Group 2 were treated with vehicle for 14 days (po, qd, from day 1 to day 14). Mice in Group 3 were treated with 30 mg/kg Cpd. 4 for 14 days (po, qd, from day 1 to day 14). Mice in Group 4 were treated with 30 mg/kg BMS986165 for 14 days (po, bid, from day 1 to day 14). The protocols and procedures involving the care and use of animals were approved by IACUC at BioDuro.

### 2. Ear Thickness Measurement

Ear thickness of mice was measured in Day 1, 3, 5, 7, 9, 11, 13 and Day 14. As seen in FIG. 20, ear thickness of mice from Group 1 (normal control) maintained at 0.28 mm during the entire experiment. Compared with G1 normal control group, after IL-23 treatment, ear swelling was observed by increased ear thickness in G2 group reaching the maximal ear thickness to 0.44 mm on day 14. Cpd. 4 showed efficacy in this mouse model of psoriasis by significantly reducing the ear thickness.

These results indicate that treatment of Cpd. 4 ameliorated the inflammation degree of IL-23-induced mice model.

### Example 8

### 1. Experimental Protocol for LPS Inhalation Induced Neutrophils Infiltration Model in C57BL/6 Mice

C57BL/6 mice (6-8 weeks old) were obtained from Shanghai LC Laboratory Animal Co. Ltd. Animals, housed and handled in a temperature-controlled environment with a 12-h light/12-h dark cycle.

A total of 60 female C57BL/6J mice will be modeled by atomization inhalation LPS 3 times a week for 10 weeks (adjustments may be made according to animal health status). LPS concentration is at 0.7 mg/mL.

On each day of challenge, animals in G1 will receive aerosol inhalation of PBS and intravenous injection of Vehicle; animals in G2 will receive aerosol inhalation of LPS and intravenous injection of Vehicle; animals in G3 will be given aerosol inhalation of LPS and intravenous injection of 50 mg/kg Cpd. X twice a week (D1/4) from week 4-10; animals in G4 will be given aerosol inhalation of LPS and intravenous injection of 12 mg/kg Cpd.XI twice a week (D1/4) from week 4-10; animals in G5 will be given aerosol inhalation of LPS and oral administration of 50 mg/kg Cpd. 4 each day from week 4-10; animals in G6 will be given aerosol inhalation of LPS and oral administration of dexamethasone 1 hour before each challenge.

### 2. BALF Neutrophils Infiltration Measurement

Lungs will be gently lavaged via the tracheal cannula with 0.8 mL of PBS containing 1% BSA and 0.6 mM EDTA 48 hr after the last LPS exposure. The procedure will be repeated twice with 0.8 mL of PBS containing 1% BSA and 0.6 mM EDTA. The bronchoalveolar lavage fluid (BALF) will be centrifuged at 300×g for 5 min at 4° C., the cell pellet will be resuspended in 1.5 mL PBS. Total numbers of neutrophils in BALF will be counted using a hemocytometer.

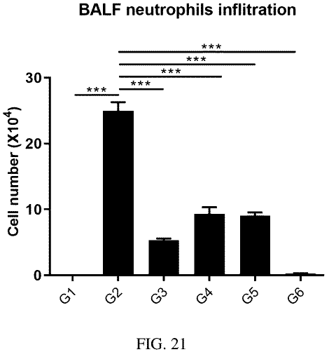

As seen in FIG. 21, compared with G1 normal control group, after LPS inhalation, there was a significant increase of BALF neutrophils in G2 group. Cpd. 4, Cpd. X and Cpd.XI showed efficacy in this mouse model by significantly reducing the BALF neutrophils.

### Example 10

### 1. Experimental Protocol for OXA Induced Delayed Type Hypersensitivity (DTH) Model in Balb/c Mice

A delayed-type hypersensitivity (DTH) reaction is an expression of cell-mediated immunity and plays a major role in the pathology and chronicity of many inflammatory disorders. Delayed-type hypersensitivity (DTH) reactions can be induced by allergens, including oxazolone.

Female Balb/c mice (6-8 weeks old) were obtained from Shanghai Jihui Experiment Animal Feeding Co. Ltd. Animals were housed and handled in a temperature-controlled environment with a 12-h light/12-h dark cycle. A total of 48 mice were assigned to 6 groups by randomization based on body weight.

To induce DTH, all right ears of mice except the G1 sham control group were applied with oxazolone (OXA). Briefly, skin inflammation was induced by topical administration with 100 μL of 1.5% OXA dissolved in acetone/olive oil on day 1 and repeated 20 μL of 1% OXA on day 6. G1 sham control group was treated same volume of vehicles. Therapeutic group of 100 mg/kg Cpd. 4 (po, 0 h/6 h after challenge on day 6) or 0.05 mg/ear dexamethasone (topical, 1 h/6 h after challenge on day 6) were applied to the mice. The protocols and procedures involving the care and use of animals were approved by the Institutional Animal Care and Use Committee (IACUC) at Chempartner (Shanghai, China).

TABLE 9

| | | | Number |
|---|---|---|---|
| Group | Treatment | Dosage | of mice |
| Group 1 (G1) | Sham control | po, 1 h/6 h after challenge | 8 |
| Group 2 (G2) | Vehicle | po, qd, day 0 to day 6 (7 D) | 8 |
| Group 3 (G3) | Cpd. 4 | 100 mg/kg, po, 0 h/6 h after | 8 |
| | | challenge on day 6 | |
| Group 4 (G4) | Dexamethasone | 0.05 mg/ear, topical, 1 h/6 h | 8 |
| | | after challenge on day 6 | |

### 2. Increase of Ear Thickness

Ear thickness were measured with a micrometer before challenge on day 6 and 24 h after oxazolone challenge on Day 7 and reported as the mean change in ear thickness. Increase of ear thickness=Ear thickness on day 7−ear thickness on day 6.

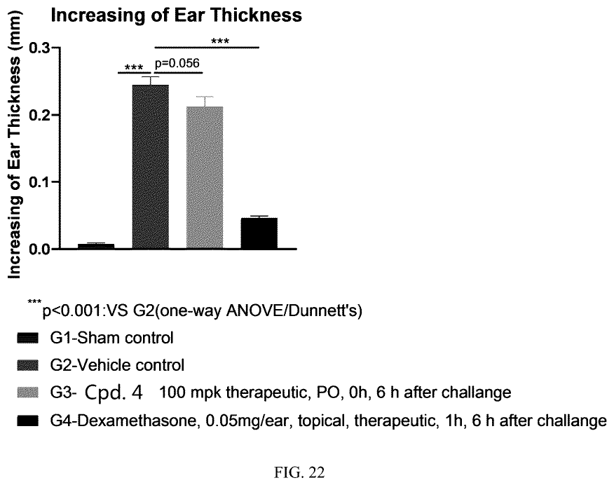

As seen in FIG. 22, there was no increase of ear thickness of mice from Group 1 (normal control) indicating no ear swelling. Compared with G1 normal control group, after OXA challenge, ear swelling was observed indicated by increase of ear thickness in G2 group by 0.24±0.01 mm. Treatment with dexamethasone showed efficacy. It should be noted that the dose for dexamethasone was far above its clinically relevant dose. Cpd. 4 showed a trend to reduce the increase of ear thickness to 0.22±0.03 mm.

1. A method for the prevention and/or treatment of an immune disease in a subject, comprising administering to the subject an effective amount of a therapeutic agent, wherein the immune disease is autoimmune disease, allergic disease or immune-mediated inflammatory disease, and the therapeutic agent is a compound of formula I, X or XI, or a pharmaceutically acceptable salt or solvate thereof, 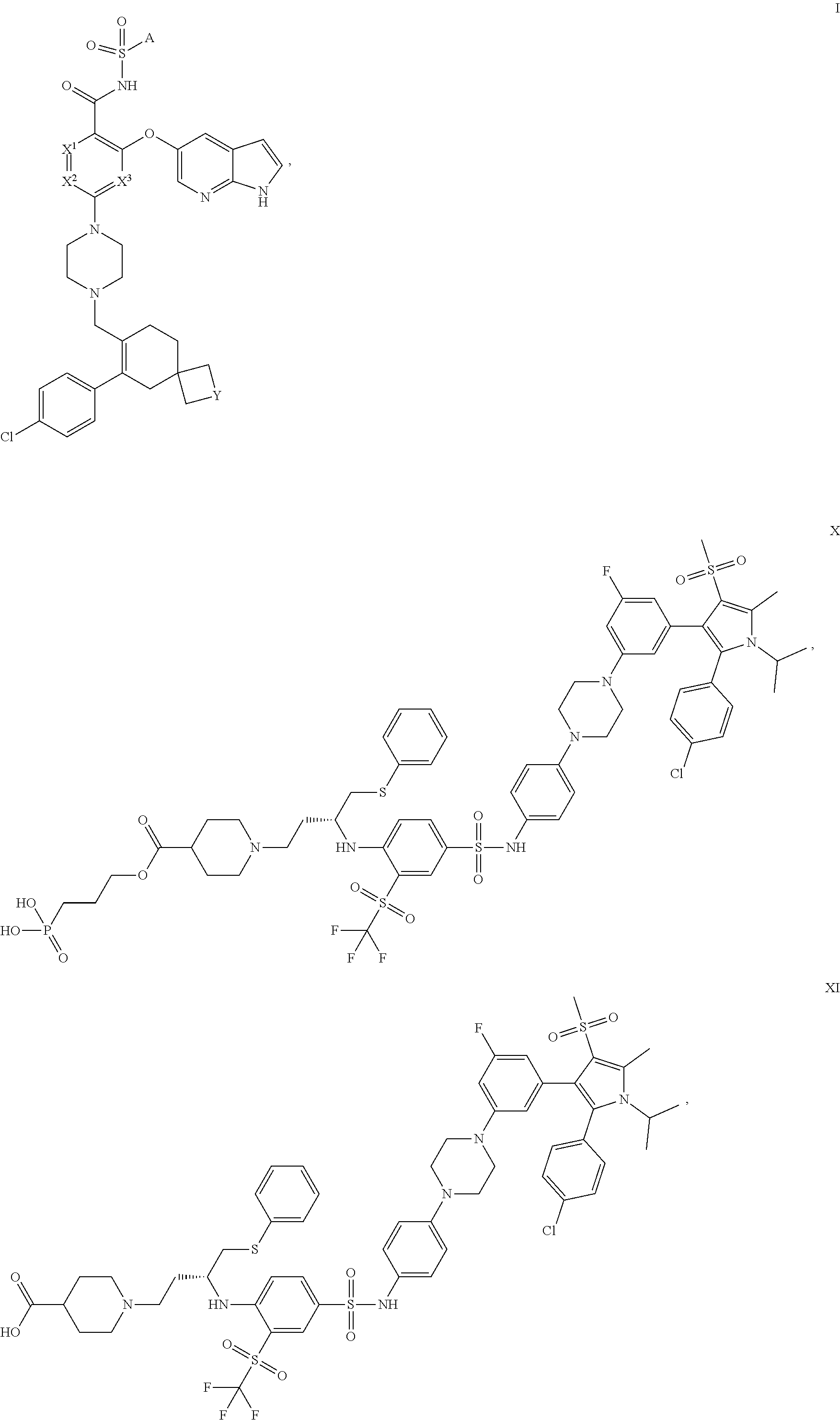
wherein, in the formula I,
A is 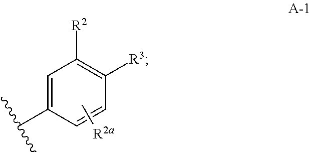
X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and
Y selected from the group consisting of —CH₂— and —O—.

2. The method as defined in claim 1, wherein the immune disease is autoimmune disease.

3. The method as defined in claim 1, wherein the immune disease is allergic disease.

4. The method as defined in claim 1, wherein the immune disease is immune-mediated inflammatory disease.

5. The method as defined in claim 1, wherein the immune disease is selected from the group consisting of acute disseminated encephalomyelitis (ADEM), Addison disease, ankylosing spondylitis, antiphospholipid syndrome (APGS), autoimmune haemolytic anaemia (AIHA), autoimmune hepatitis (AIH), autoimmune hypoparathyroidism, Autoimmune hypophysitis, autoimmune myocardioptis, autoimmune oophoritis, autoimmune orchitis, Autoimmune thrombocytopenia purpura (AITP), Behcet's disease, bullous pemphigoid, Chronic inflammatory demyelinating polyneuropathy, Churg-Strauss syndrome, Crohn's disease, dermatomyositis, familial dysautonomia, epidermolysis bullosa, Pemphigoid during pregnancy, giant cell arteritis, Goodpasture syndrome, Granulomatous disease with polyvasculitis, Graves' disease, Guillain-barre syndrome, Hashimoto Disease, Immunoglobulin A (IgA) neurological disease, inflammatory bowel disease, ulcerative colitis, interstitial cystitis (IC), Kawasaki Disease, Lambert-Eaton myasthenic syndrome (LEMS), Chronic Lyme disease, Mooren's ulcer, morphea, myasthenia gravis, neuromyotonia, Clonic syndrome of strabismus, optic neuritis, Ord thyroiditis, pemphigus, pernicious anemia, polyarteritis, polyarthritis, Polyglandular autoimmune syndrome, primary biliary cirrhosis, psoriasis, Reiter's syndrome, Sarcoidosis, rheumatic arthritis, Sjogren's syndrome, stiff-man syndrome, Takayasu arthritis, Vogt-Kovangai-Harada disease, asthma, atopic dermatitis, allergic rhinitis, food allergy, acute urticaria, and Contact dermatitis, chronic obstructive pulmonary disease.

6. The method as defined in claim 1, wherein the immune disease is Crohn's disease.

7. The method as defined in claim 1, wherein the immune disease is ulcerative colitis.

8. The method as defined in claim 1, wherein the immune disease is atopic dermatitis.

9. The method as defined in claim 1, wherein the immune disease is asthma.

10. The method as defined in claim 1, wherein the immune disease is psoriasis.

11. The method as defined in claim 1, wherein the immune disease is chronic obstructive pulmonary disease.

12. The method as defined in claim 1, wherein the immune disease is Type IV hypersensitivity disease.

13. A method for the prevention and/or inhibition of autoimmune response or overactive immune response in a subject, comprising administering to the subject an effective amount of a therapeutic agent, wherein the therapeutic agent is a compound of formula I, X or XI, or a pharmaceutically acceptable salt or solvate thereof: 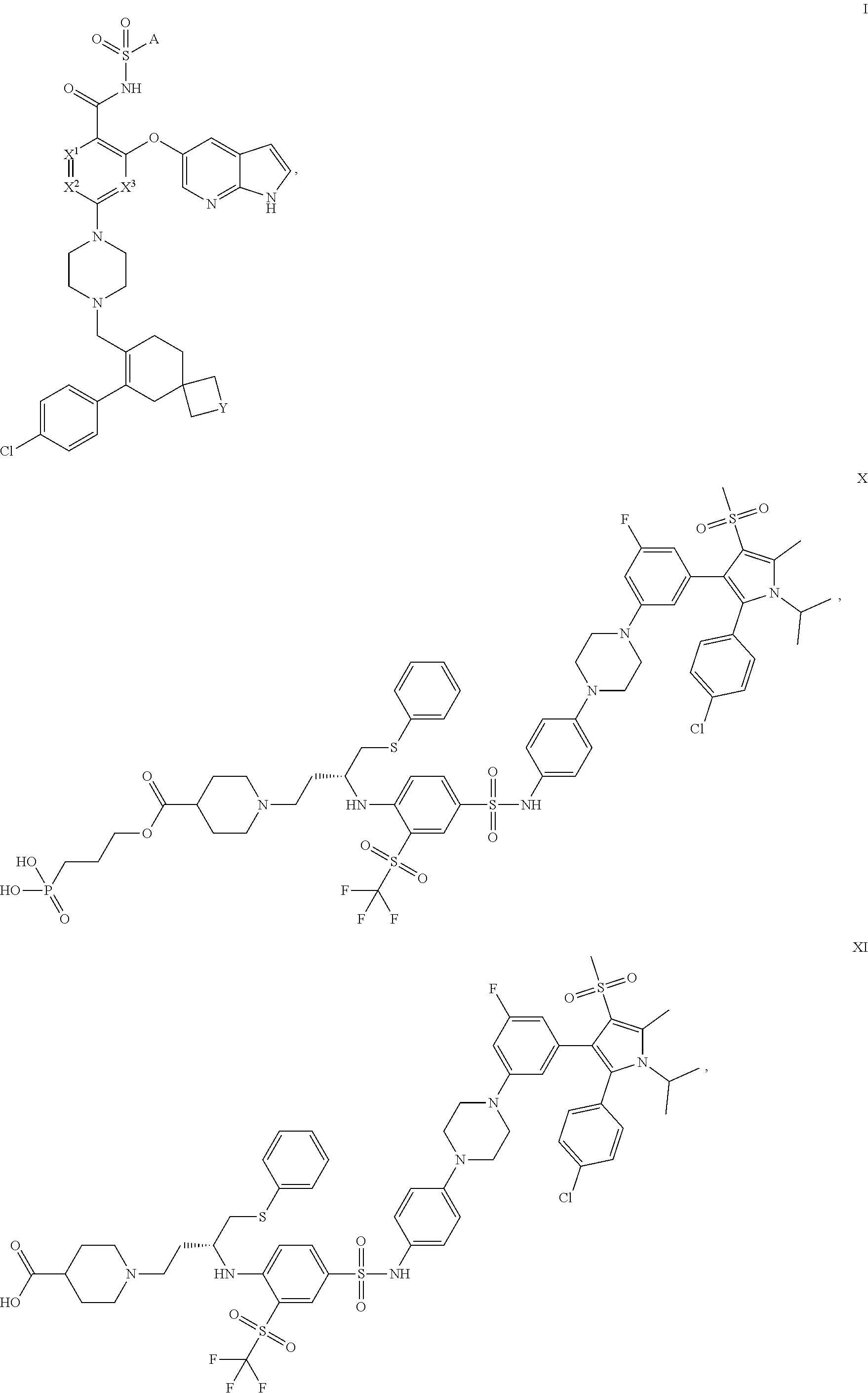
wherein, in the formula I,
A is 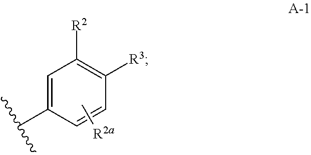
X¹, X², and X³ are each independently selected from the group consisting of —CR⁸═ and —N═;
R⁸ is selected from the group consisting of hydrogen and halogen;
R² is selected from the group consisting of —NO₂, —SO₂CH₃, and —SO₂CF₃;
R^{2a} is selected from the group consisting of hydrogen and halogen;
R³ is selected from the group consisting of hydrogen, —CN, —C≡CH, and —N(R^{4a})(R^{4b});
R^{4a} is selected from the group consisting of optionally substituted C₁₋₆ alkyl, optionally substituted C₃₋₆ cycloalkyl, heterocyclo, heteroalkyl, (cycloalkyl)alkyl, and (heterocyclo)alkyl;
R^{4b} is selected from the group consisting of hydrogen and C₁₋₄ alkyl; and
Y selected from the group consisting of —CH₂— and —O—.

14. The method as defined in claim 13, wherein the autoimmune response or overactive immune response is characterized by below feature a), b), c), d) or a combination thereof:
a) elevated neutrophils, monocytes, macrophages and/or natural killer cells;
b) elevated activated Th1 cells, and/or effector cells;
c) elevated eosinophils and/or IgE production;
d) elevated activated Th17 cells and/or neutrophils.

15. The method as defined in any one of claims 1-14, wherein the therapeutic agent is the compound of formula I, or a pharmaceutically acceptable salt or solvate thereof.

16. The method as defined in any one of claims 1-15, wherein, in the formula I,
X¹, X², and X³ are each —CH═;
or, X¹ is —CF═, and X² and X³ are each —CH═;
or, X¹ and X³ are each —CH═, and X² is —CF═;
or, X¹ and X² are each —CH═, and X³ is —CF═;
or, X¹ is —N═, and X² and X³ are each —CH═;
or, X¹ and X³ are each —CH═, and X² is —N═;
or, X¹ and X² are each —CH═, and X³ is —N═;
or, Y is —O—;
or, Y is —CH₂—;
or, R² is —NO₂;
or, R^{4a} is selected from the group consisting of:

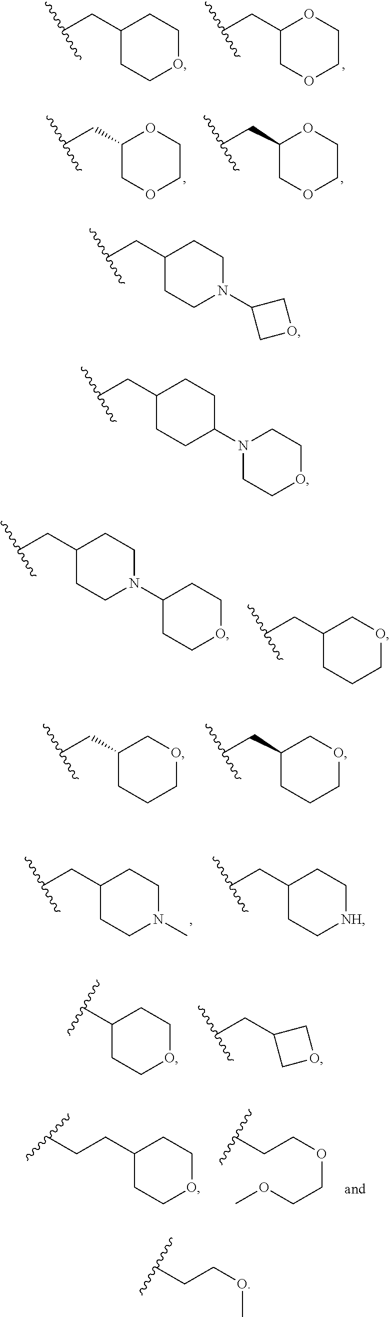

17. The method as defined in any one of claims 1-16, wherein the formula I is Formula II: 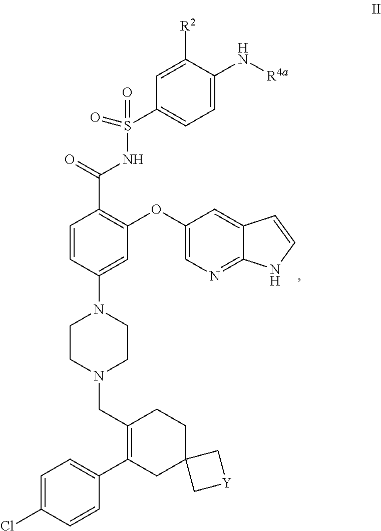
wherein Y selected from the group consisting of —CH₂— and —O—, and R² and R^{4a} are as defined in claim 1 or 15.

18. The method as defined in any one of claims 1-17, wherein R^{4a} is selected from the group consisting of:

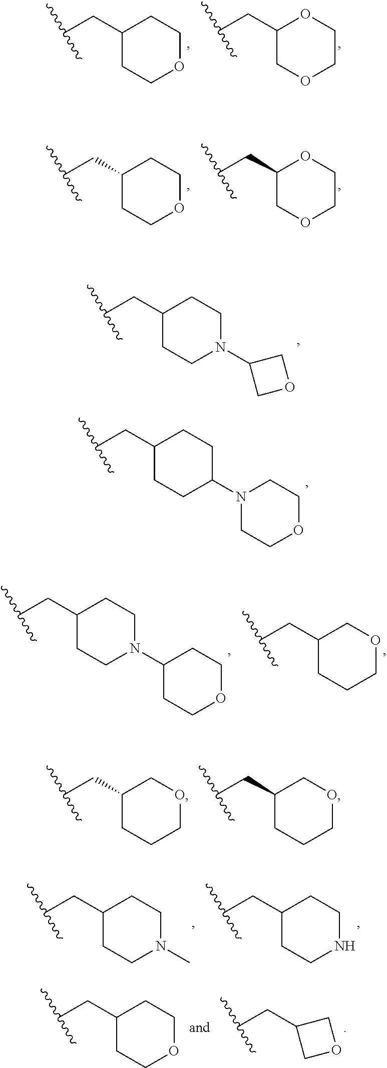

19. The method as defined in any one of claims 1-6 and 18, wherein the formula I is Formula III: 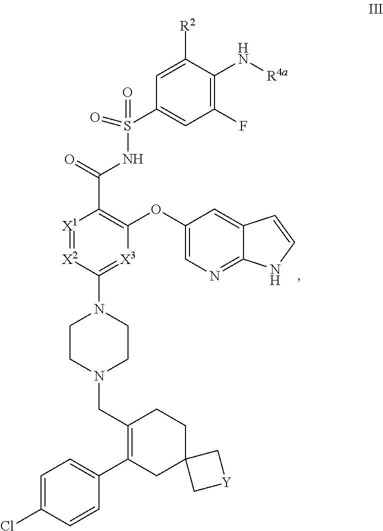
wherein Y selected from the group consisting of —CH₂— and —O—, and X¹, X², X³, R², and R^{4a} are as defined in claim 1, 15, or 17.

20. The method as defined in any one of claims 1-16 and 18, wherein the formula I is Formula IV: 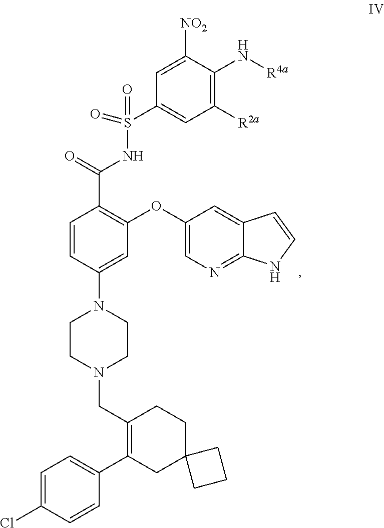
wherein R^{2a} is hydrogen or fluoro and R^{4a} is as defined in claim 1, 15, or 17.

21. The method as defined in any one of claims 1-15, wherein the compound of formula I is selected from one or more of the compounds in Table 1.

22. The method as defined in any one of claims 1-15, wherein the compound of formula I is selected from one or more of:

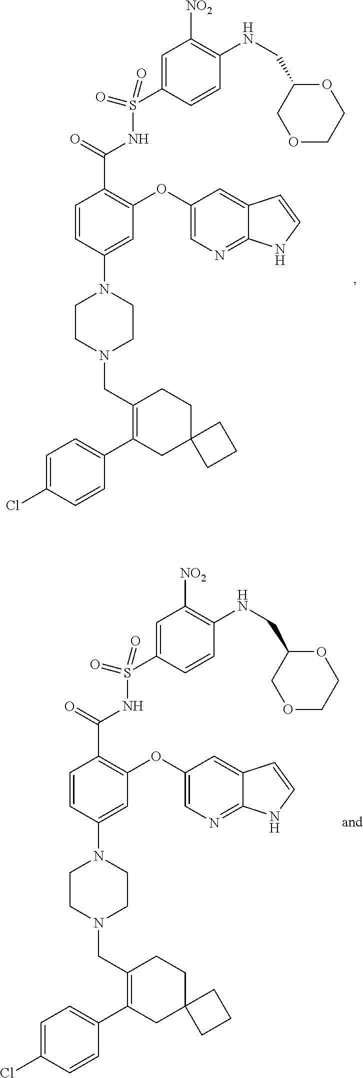 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1 ; O=C(NS(=O)(=O)c1ccc(NC[C@@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

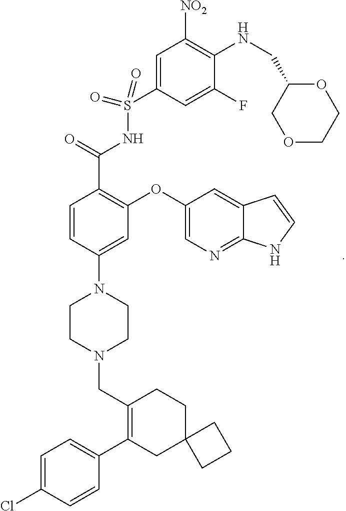 [SMILES: O=C(NS(=O)(=O)c1cc(F)c(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

23. The method as defined in any one of claims 1-15, wherein the compound of formula I is

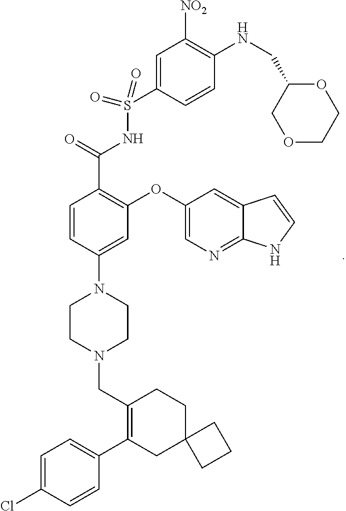 [SMILES: O=C(NS(=O)(=O)c1ccc(NC[C@H]2COCCO2)c([N+](=O)[O-])c1)c1ccc(N2CCN(CC3=C(c4ccc(Cl)cc4)CC4(CCC4)CC3)CC2)cc1Oc1cnc2[nH]ccc2c1]

24. The method as defined in any one of claims 1-14, wherein the therapeutic agent is the compound of formula X, or a pharmaceutically acceptable salt or solvate thereof.

25. The method as defined in any one of claims 1-14, wherein the therapeutic agent is the compound of formula XI, or a pharmaceutically acceptable salt or solvate thereof.

26. The method as defined in any one of claims 1-25, wherein the therapeutic agent is administered orally.

27. The method as defined in any one of claims 1-25, wherein the therapeutic agent is administered by injection.
